(19) United States
(12) Patent Application Publication
(10) Pub. No.: US 2016/0264568 A1
(43) Pub. Date: Sep. 15, 2016

(54) SUBSTITUTED TRIAZOLOPYRIDINES HAVING ACTIVITY AS MPS-1 INHIBITORS

(71) Applicant: BAYER PHARMA AKTIENGESELLSCHAFT, Berlin (DE)

(72) Inventors: Volker K. SCHULZE, OT Bergf (DE); Andreas SCHALL, Wuppertal (DE); Hans BRIEM, Berlin (DE); Antje Margret WENGNER, Berlin (DE); Gerhard SIEMEISTER, Berlin (DE); Detlef STÖCKIGT, Potsdam (DE); Philip LIENAU, Berlin (DE)

(21) Appl. No.: 14/896,429

(22) PCT Filed: Jun. 3, 2014
(86) PCT No.: PCT/EP2014/061416

§ 371 Date: Dec. 7, 2015

(30) Foreign Application Priority Data
Jun. 7, 2013 (EP) 13170978.4
Sep. 27, 2013 (EP) 13186298.9

(51) Int. Cl. C07D 471/04; C07F 5/04; C07F 5/02; C07D 333/54

(52) U.S. Cl. CPC C07D 471/04; C07D 333/54; C07F 5/04; C07F 5/025

(57) ABSTRACT

The present invention relates to substituted triazolopyridine compounds of general formula (I), in which R¹, R², R³, R⁴, and R⁵ are as given in the description and in the claims, to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

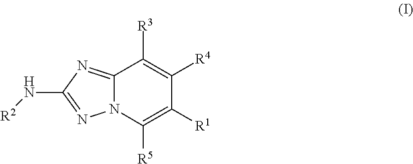

The present invention relates to substituted triazolopyridine compounds of general formula (I) as described and defined herein, to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### BACKGROUND OF THE INVENTION

The present invention relates to chemical compounds that inhibit Mps-1 (Monopolar Spindle 1) kinase (also known as Tyrosine Threonine Kinase, UK). Mps-1 is a dual specificity Ser/Thr kinase which plays a key role in the activation of the mitotic checkpoint (also known as spindle checkpoint, spindle assembly checkpoint) thereby ensuring proper chromosome segregation during mitosis [Abrieu A et al., Cell, 2001, 106, 83-93]. Every dividing cell has to ensure equal separation of the replicated chromosomes into the two daughter cells. Upon entry into mitosis, chromosomes are attached at their kinetochores to the microtubules of the spindle apparatus. The mitotic checkpoint is a surveillance mechanism that is active as long as unattached kinetochores are present and prevents mitotic cells from entering anaphase and thereby completing cell division with unattached chromosomes [Suijkerbuijk S J and Kops G J, Biochemica et Biophysica Acta, 2008, 1786, 24-31; Musacchio A and Salmon E D, Nat Rev Mol Cell Biol., 2007, 8, 379-93]. Once all kinetochores are attached in a correct amphitelic, i.e. bipolar, fashion with the mitotic spindle, the checkpoint is satisfied and the cell enters anaphase and proceeds through mitosis. The mitotic checkpoint consists of a complex network of a number of essential proteins, including members of the MAD (mitotic arrest deficient, MAD 1-3) and Bub (Budding uninhibited by benzimidazole, Bub 1-3) families, the motor protein CENP-E, Mps-1 kinase as well as other components, many of these being over-expressed in proliferating cells (e.g. cancer cells) and tissues [Yuan B et al., Clinical Cancer Research, 2006, 12, 405-10]. The essential role of Mps-1 kinase activity in mitotic checkpoint signalling has been shown by shRNA-silencing, chemical genetics as well as chemical inhibitors of Mps-1 kinase [Jelluma N et al., PLos ONE, 2008, 3, e2415; Jones M H et al., Current Biology, 2005, 15, 160-65; Dorer R K et al., Current Biology, 2005, 15, 1070-76; Schmidt M et al., EMBO Reports, 2005, 6, 866-72].

There is ample evidence linking reduced but incomplete mitotic checkpoint function with aneuploidy and tumorigenesis [Weaver B A and Cleveland D W, Cancer Research, 2007, 67, 10103-5; King R W, Biochimica et Biophysica Acta, 2008, 1786, 4-14]. In contrast, complete inhibition of the mitotic checkpoint has been recognised to result in severe chromosome missegregation and induction of apoptosis in tumour cells [Kops G J et al., Nature Reviews Cancer, 2005, 5, 773-85; Schmidt M and Medema R H, Cell Cycle, 2006, 5, 159-63; Schmidt M and Bastians H, Drug Resistance Updates, 2007, 10, 162-81].

Therefore, mitotic checkpoint abrogation through pharmacological inhibition of Mps-1 kinase or other components of the mitotic checkpoint represents a new approach for the treatment of proliferative disorders including solid tumours such as carcinomas and sarcomas and leukaemias and lymphoid malignancies or other disorders associated with uncontrolled cellular proliferation.

Different compounds have been disclosed in prior art which show an inhibitory effect on Mps-1 kinase:

WO 2009/024824 A1 discloses 2-Anilinopurin-8-ones as inhibitors of Mps-1 for the treatment of proliferate disorders. WO 2010/124826 A1 discloses substituted imidazoquinoxaline compounds as inhibitors of Mps-1 kinase. WO 2011/026579 A1 discloses substituted aminoquinoxalines as Mps-1 inhibitors.

Substituted triazolopyridine compounds have been disclosed for the treatment or prophylaxis of different diseases:

WO 2008/025821 A1 (Cellzome (UK) Ltd) relates to triazole derivatives as kinase inhibitors, especially inhibitors of ITK or PI3K, for the treatment or prophylaxis of immunological, inflammatory or allergic disorders. Said triazole derivatives are exemplified as possessing an amide, urea or aliphatic amine substituent in position 2.

WO 2009/047514 A1 (Cancer Research Technology Limited) relates to [1,2,4]-triazolo-[1,5-a]-pyridine and [1,2,4]-triazolo-[1,5-c]-pyrimidine compounds which inhibit AXL receptor tyrosine kinase function, and to the treatment of diseases and conditions that are mediated by AXL receptor tyrosine kinase, that are ameliorated by the inhibition of AXL receptor tyrosine kinase function etc., including proliferative conditions such as cancer, etc. Said compounds are exemplified as possessing a substituent in the 5-position and a substituent in the 2-position.

WO 2009/010530 A1 discloses bicyclic heterorayl compounds and their use as phosphatidylinositol (PI) 3-kinase. Among other compounds also substituted triazolopyridines are mentioned.

WO 2009/027283 A1 discloses triazolopyridine compounds and their use as ASK (apoptosis signal-regulating kinase) inhibitors for the treatment of autoimmune diseases and neurodegenerative diseases.

WO 2010/092041 A1 (Fovea Pharmaceuticals SA) relates to [1,2,4]-triazolo-[1,5-a]-pyridines, which are said to be useful as selective kinase inhibitors, to methods for producing such compounds and methods for treating or ameliorating kinase-mediated disorder. Said triazole derivatives are exemplified as possessing a 2-chloro-5-hydroxyphenyl substituent in the 6-position of the [1,2,4]-triazolo-[1,5-a]-pyridine.

WO 2011/064328 A1, WO 2011/063907 A1, WO 2011/063908 A1, and WO 2012/143329 A1 relate to [1,2,4]-triazolo-[1,5-a]-pyridines and their use for inhibition of Mps-1 kinase.

However, the state of the art described above does not specifically disclose the substituted triazolopyridine compounds of general formula (I) of the present invention, or a tautomer, an N-oxide, a hydrate, a solvate, or a salt thereof, or a mixture of same, as described and defined herein, and as hereinafter referred to as “compounds of the present invention”, or their pharmacological activity.

### SUMMARY OF THE INVENTION

The present invention covers compounds of general formula (I):

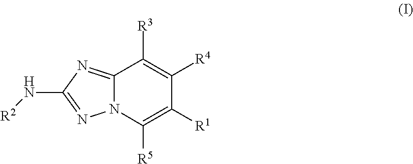

in which:
R¹ represents a phenyl- or a pyridyl- group;
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸;
R² represents a group selected from:

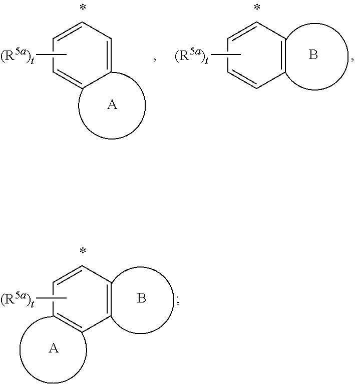

wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
B represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
each R″
   independently represents a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 10-membered heterocycloalkyl), —(CH₂)_{q}-aryl or —(CH₂)_{q}-heteroaryl;
   wherein said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —N(H)R⁸, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, p,
   represent, independently from each other, an integer of 0, 1, 2 or 3;
q represents an integer of 0, 1, 2 or 3;
and
t represents an integer of 0, 1 or 2;
   or a stereoisomer, a tautomer, an N-oxide, a hydrate, a solvate, or a salt thereof, or a mixture of same.

The present invention further relates to methods of preparing compounds of general formula (I), to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom” or “halo-” is to be understood as meaning a fluorine, chlorine, bromine or iodine atom.

The term “C₁-C₆-alkyl” is to be understood as preferably meaning a linear or branched, saturated, monovalent hydrocarbon group having 1, 2, 3, 4, 5 or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “halo-C₁-C₆-alkyl” is to be understood as preferably meaning a linear or branched, saturated, monovalent hydrocarbon group in which the term “C₁-C₆-alkyl” is defined supra, and in which one or more of the hydrogen atoms is replaced, in identically or differently, by a halogen atom. Particularly, said halogen atom is F. Said halo-C₁-C₆-alkyl group is, for example, —CF₃, —CHF₂, —CH₂F, —CF₂CF₃, or —CH₂CF₃.

The term “hydroxy-C₁-C₆-alkyl-” is to be understood as preferably meaning a linear or branched, saturated, monovalent hydrocarbon group in which the term “C₁-C₆-alkyl-” is defined supra, and in which one or more of the hydrogen atoms is replaced by a hydroxy group with the proviso that not more than one hydrogen atom attached to a single carbon atom is being replaced. Said hydroxy-C₁-C₆-alkyl- group is, for example, —CH₂OH, —CH₂CH₂—OH, —C(OH)H—CH₃, or —C(OH)H—CH₂OH.

The term “C₁-C₆-alkoxy” is to be understood as preferably meaning a linear or branched, saturated, monovalent group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said halo-C₁-C₆-alkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in identically or differently, by a halogen atom. Particularly, said halogen atom is F. Said halo-C₁-C₆-alkoxy-C₁-C₆-alkyl group is, for example, CH₂CH₂OCF₃, —CH₂CH₂OCHF₂, —CH₂CH₂OCH₂F, —CH₂CH₂OCF₂CF₃, or CH₂CH₂OCH₂CF₃.

The term “C₂-C₆-alkenyl” is to be understood as preferably meaning a linear or branched, monovalent hydrocarbon group, which contains one or more double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₆-alkynyl” is to be understood as preferably meaning a linear or branched, monovalent hydrocarbon group which contains one or more triple bonds, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₇-cycloalkyl” is to be understood as meaning a saturated, monovalent, monocyclic hydrocarbon ring which contains 3, 4, 5, 6 or 7 carbon atoms. Said C₃-C₇-cycloalkyl group is for example a cyclopropyl, cyclobutyl, cyclopentyl, cyclohexyl or cycloheptyl ring. Particularly, said ring contains 3, 4, 5 or 6 carbon atoms (“C₃-C₆-cycloalkyl”).

The term “C₄-C₈-cycloalkenyl” is to be understood as preferably meaning a monovalent, monocyclic hydrocarbon ring which contains 4, 5, 6, 7 or 8 carbon atoms and one or two double bonds, in conjugation or not, as the size of said cycloalkenyl ring allows. Particularly, said ring contains 4, 5 or 6 carbon atoms (“C₄-C₆-cycloalkenyl”). Said C₄-C₈-cycloalkenyl group is for example a cyclobutenyl, cyclopentenyl, or cyclohexenyl group.

The term “heterocyclic ring”, as used in the term “4-, 5- or 6-membered heterocyclic ring”, or “4- to 6-membered heterocyclic ring” or “4- to 5-membered heterocyclic ring”, for example, as used in the definition of compounds of general formula (I) as defined herein, is to be understood as meaning a saturated, partially unsaturated or aromatic monocyclic hydrocarbon ring which contains 1, 2, 3, 4, 5 carbon atoms, and one or more heteroatom-containing groups selected from —C(═O)—, —O—, —S—, —S(═O)—, —S(═O)₂—, ═N—, —N(H)—, —N(R″)—, wherein R″ represents a C₁-C₆-alkyl, C₃-C₆-cycloalkyl, —C(═O)—(C₁-C₆-alkyl) or —C(═O)—(C₁-C₆-cycloalkyl) group.

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), 0, S, S(═O), S(═O)₂, NR^{a}, in which R^{a} represents a hydrogen atom, or a C₁-C₆-alkyl- group; it being possible for said heterocycloalkyl group to be attached to the rest of the molecule via any one of the carbon atoms or, if present, the nitrogen atom.

Particularly, said 3- to 10-membered heterocycloalkyl can contain 2, 3, 4, 5 or 6 carbon atoms, and one or more of the above-mentioned heteroatom-containing groups (a “3- to 7-membered heterocycloalkyl”), more particularly said heterocycloalkyl can contain 4, 5 or 6 carbon atoms, and one or more of the above-mentioned heteroatom-containing groups (a “4- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

The term “4- to 10-membered heterocycloalkenyl”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), 0, S, S(═O), S(═O)₂, NR^{a}, in which R^{a} represents a hydrogen atom or a C₁-C₆-alkyl- group; it being possible for said heterocycloalkenyl group to be attached to the rest of the molecule via any one of the carbon atoms or, if present, the nitrogen atom. Examples of said heterocycloalkenyl may contain one or more double bonds, e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2, 5-dihydrofuranyl, 2, 3-dihydrofuranyl, 2,5-dihydrothiophenyl, 2,3-dihydrothiophenyl, 4,5-dihydrooxazolyl, or 4H-[1,4]thiazinyl group.

The term “aryl” is to be understood as preferably meaning a monovalent, aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a biphenyl group (a “C₁₂-aryl” group), or a ring having 13 carbon atoms, (a “C₁₃-aryl” group), e.g. a fluorenyl group, or a ring having 14 carbon atoms, (a “C₁₄-aryl” group), e.g. an anthracenyl group. Preferably, the aryl group is a phenyl group.

The term “heteroaryl” is understood as preferably meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridinyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridyl includes pyridin-2-yl, pyridin-3-yl, and pyridin-4-yl; or the term thienyl includes thien-2-yl and thien-3-yl. Preferably, the heteroaryl group is a pyridinyl group.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-05, C₃-C₄, C₁-C₂, C₁-C₃, C₁-C₄, C₁-C₅, C₁-C₆; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-05, C₃-C₄, C₂-C₃, C₂-C₄, C₂-05; particularly C₂-C₃.

Further, as used herein, the term “C₃-C₇”, as used throughout this text, e.g. in the context of the definition of “C₃-C₇-cycloalkyl”, is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

As used herein, the term “PG¹” refers to a protecting group for hydroxy groups e.g. a TMS group or TBDPS group as described for example in T. W. Greene and P. G. M. Wuts in Protective Groups in Organic Synthesis, 3^{rd} edition, Wiley 1999 (TMS=trimethylsilyl, TBDPS=tert-butyldiphenylsilyl).

As used herein, the term “PG²” refers to a protecting group for amino groups e.g. a Boc group as described for example in T. W. Greene and P. G. M. Wuts in Protective Groups in Organic Synthesis, 3^{rd} edition, Wiley 1999 (Boc=tert-butyloxycarbonyl).

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

Where the plural form of the word compounds, salts, polymorphs, hydrates, solvates and the like, is used herein, this is taken to mean also a single compound, salt, polymorph, isomer, hydrate, solvate or the like.

The compounds of this invention contain one or more asymmetric centre, depending upon the location and nature of the various substituents desired.

Asymmetric carbon atoms may be present in the (R) or (S) configuration. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds of the present invention as single stereoisomers, or as any mixture of said stereoisomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, viz.:

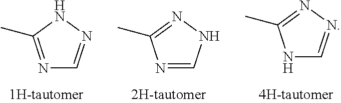

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula (I):

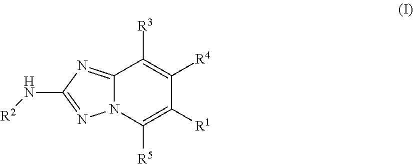

in which:
R¹ represents a phenyl- or a pyridyl- group;
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸;
R² represents:

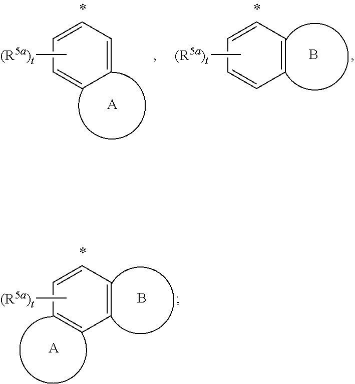

wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
B represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
each R″
   represents independently a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 1.0-membered heterocycloalkyl), —(CH₂)_{q}-aryl or —(CH₂)_{q}-heteroaryl;
   wherein said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(W)C(═O)R⁸, —N(H)C(═O)NR⁸R⁷, —N(R⁷)C(═O)NR⁸R⁷, —N(H)R⁸, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷,
   —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, p,
   represent, independently from each other, an integer of 0, 1, 2 or 3;
q represents an integer of 0, 1, 2 or 3;
and
t represents an integer of 0, 1 or 2;
   or a stereoisomer, a tautomer, an N-oxide, a hydrate, a solvate, or a salt thereof, or a mixture of same.

In a preferred embodiment, the invention relates to compounds of formula (I), wherein:
R¹ represents a phenyl group
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, C₁-C₆-alkyl-, C₁-C₆-alkoxy-, —N(H)C(═O)R⁸, —C(═O)N(H)R⁸.

In another preferred embodiment, the invention relates to compounds of formula (I), wherein:
R¹ represents a phenyl group
   which is substituted, one or more times, identically or differently, with a substituent selected from:
   R⁶—(C₁-C₆-alkoxy)-, R⁶—O—, —N(H)C(═O)R⁶, —N(H)C(═O)NR⁶R⁷, —C(═O)N(H)R⁶, —C(═O)NR⁶R⁷; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸.

In another preferred embodiment, the invention relates to compounds of formula (I), wherein:

R¹ represents a phenyl group
which is substituted, one or more times, identically or differently, with a substituent selected from:
R⁶—(C₁-C₆-alkoxy)-, R⁶—O—, —N(H)C(═O)R⁶, —N(H)C(═O)NR⁶R⁷, —C(═O)N(H)R⁶, —C(═O)NR⁶R⁷; and
which is optionally substituted, one or more times, identically or differently, with a substituent selected from:
halo-, C₁-C₆-alkyl-, C₁-C₆-alkoxy-, —N(H)C(═O)R⁸, —C(═O)N(H)R⁸.

In another preferred embodiment, the invention relates to compounds of formula (I), wherein:
R¹ represents a phenyl group
   which is substituted, one or more times, identically or differently, with a substituent selected from:
   -N(H)C(═O)R⁶, —C(═O)N(H)R⁶; and
   which is optionally substituted, one or more times, identically or differently, with a substituent selected from:
   halo-, C₁-C₆-alkyl-, C₁-C₆-alkoxy-, —N(H)C(═O)R⁸, —C(═O)N(H)R⁸.

In another preferred embodiment, the invention relates to compounds of formula (I), wherein:
R¹ represents a phenyl group
   which is substituted, one or more times, identically or differently, with a substituent selected from:
   R⁶—(C₁-C₆-alkoxy)-, R⁶—O—, —N(H)C(═O)R⁶, —N(H)C(═O)NR⁶R⁷, —C(═O)N(H)R⁶, —C(═O)NR⁶R⁷; and
   which is optionally substituted, one or more times, identically or differently, with a substituent selected from:
   halo-, C₁-C₆-alkyl-, C₁-C₆-alkoxy-.

In another preferred embodiment, the invention relates to compounds of formula (I), wherein:
R¹ represents a phenyl group
   which is substituted, one or more times, identically or differently, with a substituent selected from:
   —N(H)C(═O)R⁶, —C(═O)N(H)R⁶; and
   which is optionally substituted, one or more times, identically or differently, with a substituent selected from:
   halo-, C₁-C₆-alkyl-, C₁-C₆-alkoxy-.

In a preferred embodiment, the invention relates to compounds of formula (I), wherein:
R¹ represents a phenyl group
   which is substituted, one or more times, identically or differently, with a —N(H)C(═O)R⁶ substituent, and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸.

In a preferred embodiment, the invention relates to compounds of formula (I), wherein:
R¹ represents a phenyl group
   which is substituted, one or more times, identically or differently, with a —C(═O)N(H)R⁶ substituent, and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸.

In a preferred embodiment, the invention relates to compounds of formula (I), wherein:
R¹ represents a phenyl group
   which is substituted, one or more times, identically or differently, with a —N(H)C(═O)R⁶ substituent, and
   which is optionally substituted, one or more times, identically or differently, with a substituent selected from:
   halo-, C₁-C₆-alkyl-, C₁-C₆-alkoxy-.

In a preferred embodiment, the invention relates to compounds of formula (I), wherein:
R¹ represents a phenyl group
   which is substituted, one or more times, identically or differently, with a —C(═O)N(H)R⁶ substituent, and
   which is optionally substituted, one or more times, identically or differently, with a substituent selected from:
   halo-, C₁-C₆-alkyl-, C₁-C₆-alkoxy-.

In another preferred embodiment, the invention relates to compounds of formula (I), wherein:
R¹ represents a phenyl group
   which is substituted, in para-position to the point of attachment of the phenyl group with the rest of the molecule, with —N(H)C(═O)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸.

In another preferred embodiment, the invention relates to compounds of formula (I), wherein:
R¹ represents a phenyl group
   which is substituted, in para-position to the point of attachment of the phenyl group with the rest of the molecule, with —C(═O)N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸.

In another preferred embodiment, the invention relates to compounds of formula (I), wherein:
R¹ represents a phenyl group which is para-substituted with respect to the point of attachment of the phenyl group with the rest of the molecule, as depicted in formula (I), with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
      which is optionally substituted, one or more times, identically or differently, with a substituent selected from:
   halo-, C₁-C₆-alkyl-, C₁-C₆-alkoxy-, —N(H)C(═O)R⁸, —C(═O)N(H)R⁸.

In another preferred embodiment, the invention relates to compounds of formula (I), wherein:
R¹ represents a phenyl group which is para-substituted with respect to the point of attachment of the phenyl group with the rest of the molecule, as depicted in formula (I), with a substituent selected from:
   R⁶—(C₁-C₆-alkoxy)-, R⁶—O—, —N(H)C(═O)R⁶, —N(H)C(═O)NR⁶R⁷, —C(═O)N(H)R⁶, —C(═O)NR⁶R⁷; and
      which is optionally substituted, one or more times, identically or differently, with a substituent selected from:
   halo-, C₁-C₆-alkyl-, C₁-C₆-alkoxy-, —N(H)C(═O)R⁸, —C(═O)N(H)R⁸.

In another preferred embodiment, the invention relates to compounds of formula (I), wherein:
R¹ represents a phenyl group which is para-substituted with respect to the point of attachment of the phenyl group with the rest of the molecule, as depicted in formula (I), with a substituent selected from:
   —N(H)C(═O)R⁶, —C(═O)N(H)R⁶; and
      which is optionally substituted, one or more times, identically or differently, with a substituent selected from:
   halo-, C₁-C₆-alkyl-, C₁-C₆-alkoxy-, —N(H)C(═O)R⁸, —C(═O)N(H)R⁸.

In another preferred embodiment, the invention relates to compounds of formula (I), wherein:
R¹ represents a phenyl group which is para-substituted with respect to the point of attachment of the phenyl group with the rest of the molecule, as depicted in formula (I), with a substituent selected from:
   —N(H)C(═O)R⁶, —C(═O)N(H)R⁶; and
      which is optionally substituted, one or more times, identically or differently, with a substituent selected from:
   halo-, C₁-C₆-alkyl-, C₁-C₆-alkoxy-.

In another preferred embodiment, the invention relates to compounds of formula (I), wherein:
R¹ represents

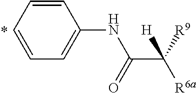

wherein * indicates the point of attachment of said group with the rest of the molecule;
wherein R^{6a} is a phenyl- group which is optionally substituted, one or more times, identically or differently, with a substituent selected from: halo-, methyl-, methoxy-; and
wherein R⁹ represents a group selected from:
C₁-C₃-alkyl-, hydroxy-C₁-C₃-alkyl-, —N(R¹⁰)R¹⁰; —C₁-C₂-alkyl-N(R¹⁰)R¹⁰; in which R¹⁰ represents a hydrogen atom or a methyl- group.

In another preferred embodiment, the invention relates to compounds of formula (I), wherein:
R¹ represents a group selected from:

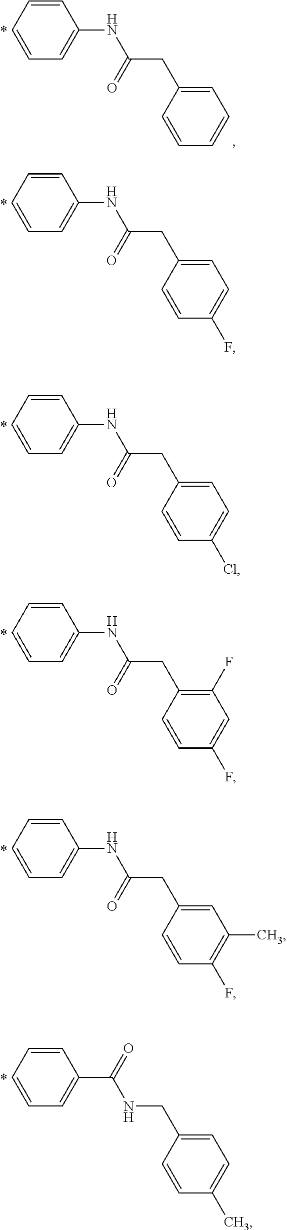

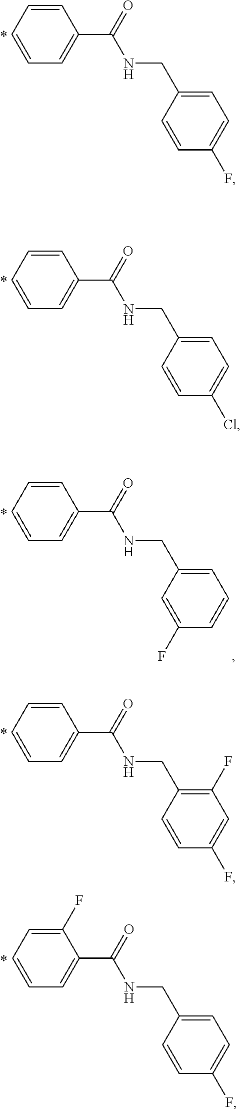

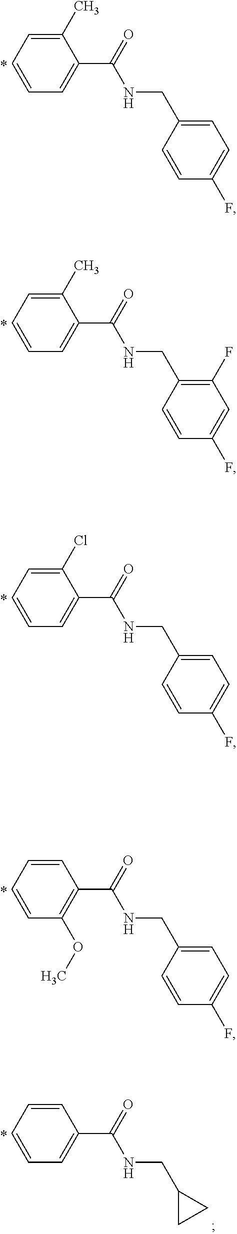

wherein * indicates the point of attachment of said group with the rest of the molecule.

In another preferred embodiment, the invention relates to compounds of formula (I), supra, wherein R¹ represents a group selected from:

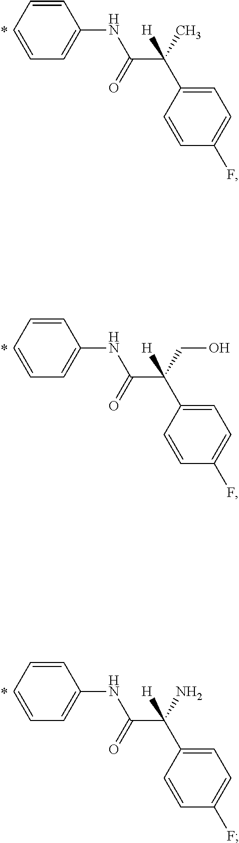

wherein * indicates the point of attachment of said groups with the rest of the molecule.

In another preferred embodiment, the invention relates to compounds of formula (I), supra, wherein R¹ represents

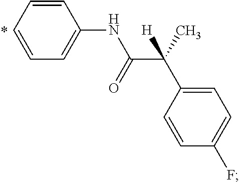

wherein * indicates the point of attachment of said groups with the rest of the molecule.

In another preferred embodiment, the invention relates to compounds of formula (I), supra, wherein R¹ represents

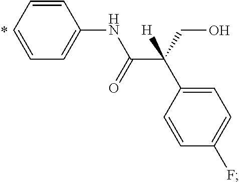

wherein * indicates the point of attachment of said groups with the rest of the molecule.

In another preferred embodiment, the invention relates to compounds of formula (I), supra, wherein R¹ represents

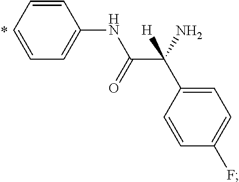

wherein * indicates the point of attachment of said groups with the rest of the molecule.

In another preferred embodiment, the invention relates to compounds of formula (I), wherein A represents a 5- to 6-membered heterocyclic ring; which is optionally substituted, one or more times, identically or differently, with halo-, —CN, —OH, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R⁸—(C₁-C₃-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—.

In another preferred embodiment, the invention relates to compounds of formula (I), wherein A represents a 5- to 6-membered heterocyclic ring; which is optionally, one or more times, identically or differently, substituted with C₁-C₃-alkyl-, halo-C₁-C₃-alkyl-.

In another preferred embodiment, the invention relates to compounds of formula (I), wherein A represents a 5- to 6-membered heterocyclic ring.

In another preferred embodiment, the invention relates to compounds of formula (I), wherein A represents a 5-membered heterocyclic ring.

In another preferred embodiment, the invention relates to compounds of formula (I), wherein B represents a 5- to 6-membered heterocyclic ring; which is optionally substituted, one or more times, identically or differently, with halo-, —CN, —OH, C₁-C₃-alkyl-, halo-C₁-C₃-alkyl-, C₁-C₃-alkoxy-, halo-C₁-C₃-alkoxy-, hydroxy-C₁-C₃-alkyl-, C₁-C₃-alkoxy-C₁-C₃-alkyl-, halo-C₁-C₃-alkoxy-C₁-C₃-alkyl-, R⁸—(C₁-C₃-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—.

In another preferred embodiment, the invention relates to compounds of formula (I), wherein B represents a 5- to 6-membered heterocyclic ring; which is optionally, one or more times, identically or differently, substituted with C₁-C₃-alkyl-, halo-C₁-C₃-alkyl-.

In another preferred embodiment, the invention relates to compounds of formula (I), wherein B represents a 5- to 6-membered heterocyclic ring.

In another preferred embodiment, the invention relates to compounds of formula (I), wherein B represents a 5-membered heterocyclic ring.

In another preferred embodiment, the invention relates to compounds of formula (I), wherein R² represents:

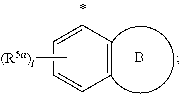

wherein * indicates the point of attachment of said group with the rest of the molecule.

In another preferred embodiment, the invention relates to compounds of formula (I), wherein R² represents:

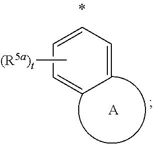

wherein * indicates the point of attachment of said group with the rest of the molecule.

In another preferred embodiment, the invention relates to compounds of formula (I), wherein R² represents:

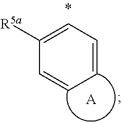

wherein * indicates the point of attachment of said group with the rest of the molecule.

In another preferred embodiment, the invention relates to compounds of formula (I), wherein R² is selected from:

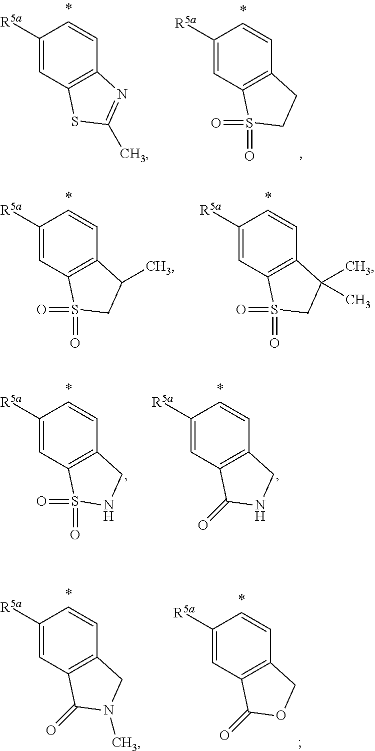

wherein * indicates the point of attachment of said groups with the rest of the molecule.

In another preferred embodiment, the invention relates to compounds of formula (I), wherein R² is selected from:

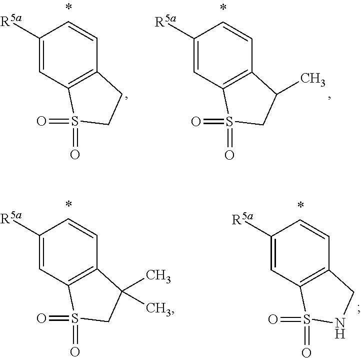

wherein * indicates the point of attachment of said groups with the rest of the molecule.

In another preferred embodiment, the invention relates to compounds of formula (I), wherein R² is selected from:

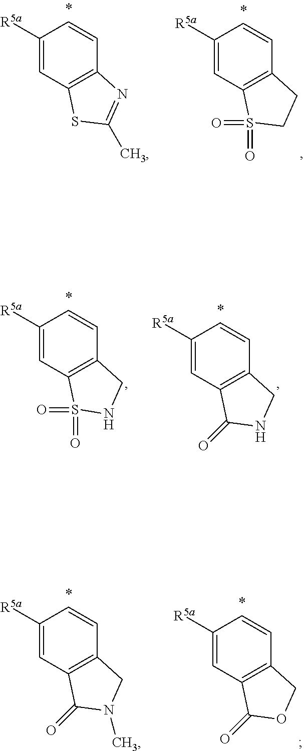

wherein * indicates the point of attachment of said groups with the rest of the molecule.

In another preferred embodiment, the invention relates to compounds of formula (I), wherein R² represents:

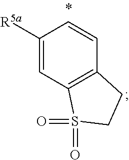

wherein * indicates the point of attachment of said groups with the rest of the molecule.

In another preferred embodiment, the invention relates to compounds of formula (I), supra, wherein R⁵ represents a hydrogen atom or a methyl- group.

In another preferred embodiment, the invention relates to compounds of formula (I), supra, wherein R⁵ represents a hydrogen atom.

In another preferred embodiment, the invention relates to compounds of formula (I), wherein:

t=1; and

R^{5a} represents a group selected from:
halo-, C₁-C₆-alkyl-, C₁-C₆-alkoxy-, halo-C₁-C₆-alkoxy-,
hydroxy-C₁-C₆-alkyl-, C₁-C₆-alkoxy-C₁-C₆-alkyl-,
halo-C₁-C₆-alkoxy-C₁-C₆-alkyl-, R⁸—(C₁-C₆-alkoxy)-, R⁸—O—, R⁸—S—, R⁸—S(═O)₂—, (C₃-C₆-cycloalkyl)-(CH₂)ₙ—O—.

Preferably, R^{5a} is selected from:
halo-, C₁-C₆-alkyl-, C₁-C₆-alkoxy-, halo-C₁-C₆-alkoxy-, C₁-C₆-alkoxy-C₁-C₆-alkyl-,
(C₃-C₆-cycloalkyl)-(CH₂)ₙ—O—.

More preferably, R^{5a} is selected from:
F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—,
CF₃CH₂—O—.

In another preferred embodiment, the invention relates to compounds of formula (I), wherein:

t=1; and

R^{5a} represents a C₁-C₆-alkoxy- group.

In another preferred embodiment, the invention relates to compounds of formula (I), wherein:

t=1; and

R^{5a} represents a C₁-C₃-alkoxy- group.

In another preferred embodiment, the invention relates to compounds of formula (I), wherein:

t=1; and

R^{5a} represents a halo-C₁-C₆-alkoxy- group.

In another preferred embodiment, the invention relates to compounds of formula (I), wherein:

t=1; and

R^{5a} represents a halo-C₁-C₃-alkoxy- group.

In another preferred embodiment, the invention relates to compounds of formula (I), wherein:

t=1; and

R^{5a} represents a (C₃-C₆-cycloalkyl)-(CH₂)ₙ—O— group.

In another preferred embodiment, the invention relates to compounds of formula (I), supra, wherein t=1, and R^{5a} represents a group selected from: C₁-C₃-alkoxy-, halo-C₁-C₃-alkoxy-, C₁-C₃-alkyl-.

In another preferred embodiment, the invention relates to compounds of formula (I), supra, wherein t=1, and R^{5a} represents a group selected from: C₁-C₂-alkoxy-, halo-C₁-C₂-alkoxy-, C₁-C₂-alkyl-.

In another preferred embodiment, the invention relates to compounds of formula (I), supra, wherein t=1, and R^{5a} represents a group selected from:

C₁-C₃-alkoxy-, halo-C₁-C₃-alkoxy-.

In another preferred embodiment, the invention relates to compounds of formula (I), supra, wherein t=1, and R^{5a} represents a group selected from:

C₁-C₂-alkoxy-, halo-C₁-C₂-alkoxy-.

In another preferred embodiment, the invention relates to compounds of formula (I), supra, wherein t=1, and R^{5a} represents a methoxy- or ethoxy-group which is optionally substituted, one or more times, identically or differently, with a halogen atom. The preferred halogen atom is F.

In another preferred embodiment, the invention relates to compounds of formula (I), supra, wherein t=1, and R^{5a} represents a group selected from: methoxy-, ethoxy-, F₃C—CH₂—O—.

In another preferred embodiment, the invention relates to compounds of formula (I), supra, wherein t=1, and R^{5a} represents a group selected from: methoxy-, F₃C—CH₂—O—.

In another preferred embodiment, the invention relates to compounds of formula (I), supra, wherein t=1, and R^{5a} represents methoxy-.

In another preferred embodiment, the invention relates to compounds of formula (I), supra, wherein t=1, and R^{5a} represents ethoxy-.

In another preferred embodiment, the invention relates to compounds of formula (I), supra, wherein t=1, and R^{5a} represents F₃C—CH₂—O—.

In another preferred embodiment, the invention relates to compounds of formula (I), wherein:

R⁶ represents a group selected from:
C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 10-membered heterocycloalkyl), —(CH₂)_{q}-aryl, or —(CH₂)_{q}-heteroaryl;
wherein said group is optionally substituted, one or more times, identically or differently, with a substituent selected from:
halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-;
wherein q is 1 or 2.

In another preferred embodiment, the invention relates to compounds of formula (I), wherein:

R⁶ represents a group selected from:
—(CH₂)_{q}—(C₃-C₆-cycloalkyl), —(CH₂)_{q}-(3- to 10-membered heterocycloalkyl), —(CH₂)_{q}-aryl, or —(CH₂)_{q}-heteroaryl;
wherein said group is optionally substituted, one or more times, identically or differently, with a substituent selected from:
halo-, C₁-C₆-alkyl-;
wherein q is 0 or 1.

The C₃-C₆-cycloalkyl- group preferably is a cyclopropyl- group; the aryl-group is preferably a phenyl- group; the heteroaryl- group is preferably a pyridyl- group.

In another preferred embodiment, the invention relates to compounds of formula (I), wherein:

R⁶ represents a group selected from:
—(CH₂)—(C₃-C₆-cycloalkyl), —(CH₂)-aryl;
wherein said group is optionally substituted, one or more times, identically or differently, with a substituent selected from:
halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-.
The C₃-C₆-cycloalkyl- group preferably is a cyclopropyl- group; the aryl-group is preferably a phenyl- group.

In another preferred embodiment, the invention relates to compounds of formula (I), wherein:

R⁶ represents a group selected from:
—CH₂—(C₃-C₆-cycloalkyl) or —CH₂-aryl;
wherein said group is optionally substituted, one or more times, identically or differently, with a substituent selected from:
halo-, C₁-C₆-alkyl-, halo-C₁-C₆-alkyl-, halo-C₁-C₆-alkoxy-.
The C₃-C₆-cycloalkyl- group preferably is a cyclopropyl- group; the aryl-group is preferably a phenyl- group.

In another preferred embodiment, the invention relates to compounds of formula (I), wherein:

R⁶ represents a group selected from:
—(CH₂)—(C₃-C₆-cycloalkyl), —(CH₂)-aryl;
wherein said group is optionally substituted, one or more times, identically or differently, with a substituent selected from:
halo-, C₁-C₆-alkyl-.
The C₃-C₆-cycloalkyl- group preferably is a cyclopropyl- group; the aryl-group is preferably a phenyl- group.

In another preferred embodiment, the invention relates to compounds of formula (I), wherein:

R⁶ represents a group selected from:
—(CH₂)-aryl;
wherein said group is optionally substituted, one or more times, identically or differently, with a substituent selected from:
halo-, C₁-C₆-alkyl-.
The aryl- group is preferably a phenyl- group.

In another preferred embodiment, the invention relates to compounds of formula (I), wherein:

R⁶ represents a group selected from:
—(CH₂)—(C₃-C₆-cycloalkyl);
wherein said group is optionally substituted, one or more times, identically or differently, with a substituent selected from:
halo-, C₁-C₃-alkyl-.
The C₃-C₆-cycloalkyl- group preferably is a cyclopropyl- group.

In another preferred embodiment, the invention relates to compounds of formula (I), wherein:

R⁶ represents a group selected from:
—(CH₂)-phenyl, —(CH₂)-cyclopropyl;
wherein said group is optionally substituted, one or more times, identically or differently, with a substituent selected from:
halo-, C₁-C₃-alkyl-.

In another preferred embodiment, the invention relates to compounds of formula (I), supra, wherein R⁷ represents a C₁-C₃-alkyl- group.

In another preferred embodiment, the invention relates to compounds of formula (I), supra, wherein R⁷ represents a methyl- group.

In another preferred embodiment, the invention relates to compounds of formula (I), supra, wherein R⁸ represents a hydrogen atom or a C₁-C₆-alkyl-group, wherein said C₁-C₆-alkyl- group is optionally substituted, one or more times, with a halogen atom.

In another preferred embodiment, the invention relates to compounds of formula (I), supra, wherein R⁸ represents a hydrogen atom or a C₁-C₃-alkyl-group, wherein said C₁-C₃-alkyl- group is optionally substituted, one or more times, with a halogen atom.

In another preferred embodiment, the invention relates to compounds of formula (I), supra, wherein R⁹ represents a group selected from: C₁-C₃-alkyl-, hydroxy-C₁-C₃-alkyl-, —N(R¹⁰)R¹⁰, —C₁-C₂-alkyl-N(R¹⁰)R¹⁰; in which R¹⁰ represents a hydrogen atom or a methyl- group.

In another preferred embodiment, the invention relates to compounds of formula (I), supra, wherein R⁹ represents a group selected from: methyl-, hydroxy-C₁-C₂-alkyl-, —N(R¹⁰)R¹⁰, —C₁-C₂-alkyl-N(R¹⁰)R¹⁰; in which R¹⁰ represents a hydrogen atom or a methyl- group.

In another preferred embodiment, the invention relates to compounds of formula (I), supra, wherein R⁹ represents a group selected from: methyl-, HO—CH₂—, H₂N—CH₂—, —NH₂.

In another preferred embodiment, the invention relates to compounds of formula (I), supra, wherein R⁹ represents a group selected from: methyl-, HO—CH₂—, —NH₂.

In another preferred embodiment, the invention relates to compounds of formula (I), supra, wherein R⁹ represents a methyl- group.

In another preferred embodiment, the invention relates to compounds of formula (I), supra, wherein R⁹ represents a HO—CH₂— group.

In another preferred embodiment, the invention relates to compounds of formula (I), supra, wherein R⁹ represents a —NH₂ group.

In another preferred embodiment, the invention relates to compounds of formula (I), wherein:

n represents an integer of 0, 1 or 2.

Preferably, n represent 0 or 1.

In another preferred embodiment, the invention relates to compounds of formula (I), wherein:

q represents an integer of 0, 1 or 2.

Preferably, q represents 1 or 2.

More preferably, q=1.

In another preferred embodiment, the invention relates to compounds of formula (I), wherein:

t represents an integer of 1 or 2.

Preferably, t represents 1.

It is to be understood that the present invention relates also to any combination of the preferred embodiments described above.

Some examples of combinations are given hereinafter. However, the invention is not limited to these combinations.

In a preferred embodiment, the invention relates to compounds of formula (I):

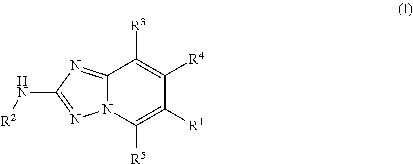

in which:
R¹ represents a phenyl- or a pyridyl- group;
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸;
R² represents:

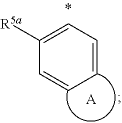

wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
R^{5a} represents a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 10-membered heterocycloalkyl), —(CH₂)_{q}-aryl or —(CH₂)_{q}-heteroaryl;
   wherein said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, p,
   represent, independently from each other, an integer of 0, 1, 2 or 3;
and
q represents an integer of 0, 1, 2 or 3;
   or a tautomer, an N-oxide, a hydrate, a solvate, or a salt thereof, or a mixture of same.

In another preferred embodiment, the invention relates to compounds of formula (I):

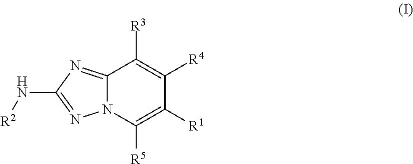

in which:
R¹ represents

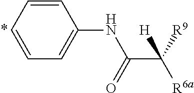

wherein * indicates the point of attachment of said group with the rest of the molecule;
R² represents:

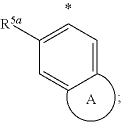

wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
R^{5a} represents a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R^{6a} represents a phenyl- group which is optionally substituted, one or more times, identically or differently, with a substituent selected from: halo-, methyl-, methoxy-;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
R⁹ represents a group selected from:
   C₁-C₃-alkyl-, hydroxy-C₁-C₃-alkyl-, —N(R¹⁰)R¹⁰, —C₁-C₂-alkyl-N(R¹⁰)R¹⁰;
R¹⁰ represents a hydrogen atom or a methyl- group;
n, m, p,
   represent, independently from each other, an integer of 0, 1, 2 or 3;
and
q represents an integer of 0, 1, 2 or 3;
   or a tautomer, an N-oxide, a hydrate, a solvate, or a salt thereof, or a mixture of same.

In another preferred embodiment, the invention relates to compounds of formula (I):

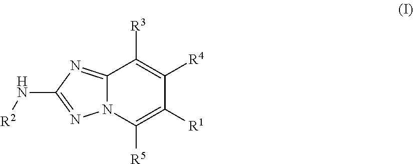

in which:
R¹ represents a phenyl- or a pyridyl- group;
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸;
R² represents a group selected from:

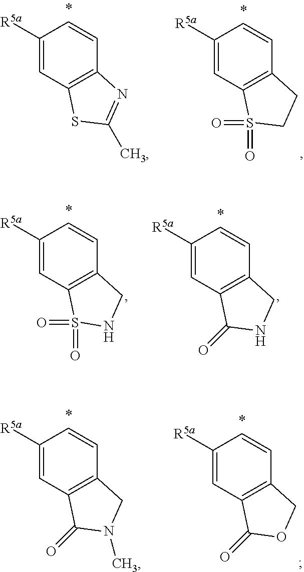

wherein * indicates the point of attachment of said groups with the rest of the molecule;
R³ represents a hydrogen atom;
R⁴ represents a hydrogen atom;
R⁵ represents a hydrogen atom or a C₁-C₃-alkyl- group;
R^{5a} represents a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R⁶ represents a group selected from:
   C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 10-membered heterocycloalkyl), —(CH₂)_{q}-aryl or —(CH₂)_{q}-heteroaryl;
   wherein said group being optionally substituted, one or more times, identically or differently, with a substituent selected from:
   halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, p,
   represent, independently from each other, an integer of 0, 1, 2 or 3;
and
q represents an integer of 0, 1, 2 or 3;
   or a tautomer, an N-oxide, a hydrate, a solvate, or a salt thereof, or a mixture of same.

In another preferred embodiment, the invention relates to compounds of formula (I):

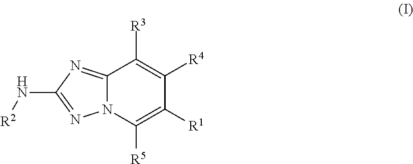

in which:
R¹ represents

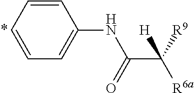

wherein * indicates the point of attachment of said group with the rest of the molecule;
R² represents a group selected from:

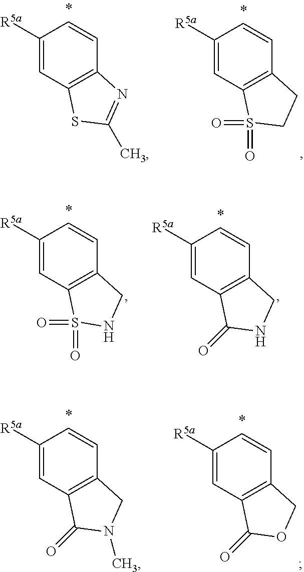

wherein * indicates the point of attachment of said groups with the rest of the molecule;
R³ represents a hydrogen atom;
R⁴ represents a hydrogen atom;
R⁵ represents a hydrogen atom or a C₁-C₃-alkyl- group;
R^{5a} represents a group selected from:
   halo-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R^{6a} represents a phenyl- group which is optionally substituted, one or more times, identically or differently, with a substituent selected from: halo-, methyl-, methoxy-;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
R⁹ represents a group selected from:
   C₁-C₃-alkyl-, hydroxy-C₁-C₃-alkyl-, —N(R¹⁰)R¹⁰, —C₁-C₂-alkyl-N(R¹⁰)R¹⁰;
R¹⁰ represents a hydrogen atom or a methyl- group;
   or a tautomer, an N-oxide, a hydrate, a solvate, or a salt thereof, or a mixture of same.

In another preferred embodiment, the invention relates to compounds of formula (I):

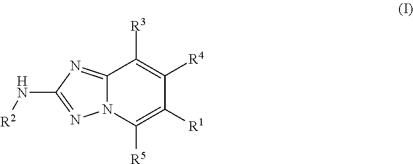

in which:
R¹ represents a phenyl- or a pyridyl- group;
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸;
R² represents:

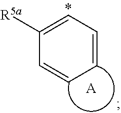

wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
R^{5a} represents a group selected from:
   F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃C H₂—O—;
R⁶ represents a group selected from:
   C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 10-membered heterocycloalkyl), —(CH₂)_{q}-aryl or —(CH₂)_{q}-heteroaryl;
   wherein said group being optionally substituted, one or more times, identically or differently, with a substituent selected from: halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, p,
   represent, independently from each other, an integer of 0, 1, 2 or 3;
and
q represents an integer of 0, 1, 2 or 3;
   or a tautomer, an N-oxide, a hydrate, a solvate, or a salt thereof, or a mixture of same.

In another preferred embodiment, the invention relates to compounds of formula (I):

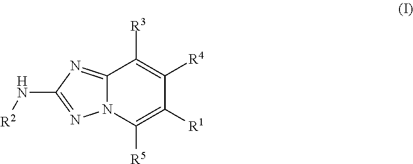

in which:
R¹ represents

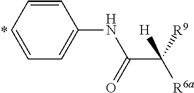

wherein * indicates the point of attachment of said group with the rest of the molecule;
R² represents:

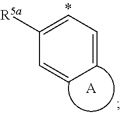

wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
R^{5a} represents a group selected from:
   F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃CH₂—O—;
R^{6a} represents a phenyl- group which is optionally substituted, one or more times, identically or differently, with a substituent selected from: halo-, methyl-, methoxy-;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
R⁹ represents a group selected from:
   C₁-C₃-alkyl-, hydroxy-C₁-C₃-alkyl-, —N(R¹⁰)R¹⁰, —C₁-C₂-alkyl-N(R¹⁰)R¹⁰;
R¹⁰ represents a hydrogen atom or a methyl- group;
   or a tautomer, an N-oxide, a hydrate, a solvate, or a salt thereof, or a mixture of same.

In another preferred embodiment, the invention relates to compounds of formula (I):

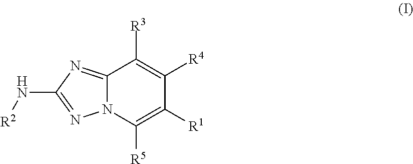

in which:
R¹ represents a phenyl- or a pyridyl- group;
   which is substituted, one or more times, identically or differently, with a substituent selected from:
   R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, —S(═O)₂N(H)R⁶; and
   which is optionally substituted, one or more times, identically or differently, with a substituent selected from:
   halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, —N(H)S(═O)₂R⁸;
R² represents a group selected from:

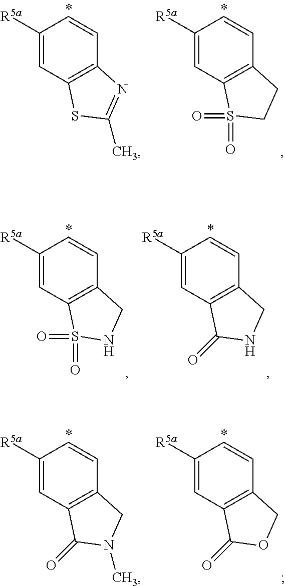

wherein * indicates the point of attachment of said groups with the rest of the molecule;
R³ represents a hydrogen atom;
R⁴ represents a hydrogen atom;
R⁵ represents a hydrogen atom or a C₁-C₃-alkyl- group;
R^{5a} represents a group selected from:
   F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃CH₂—O—;
R⁶ represents a group selected from:
   C₃-C₆-cycloalkyl-, 3- to 10-membered heterocycloalkyl-, aryl-, heteroaryl-, —(CH₂)_{q}—(C₃-C₆-cycloalkyl), —(CH₂)_{q}-(3- to 10-membered heterocycloalkyl), —(CH₂)_{q}-aryl or —(CH₂)_{q}-heteroaryl;
   wherein said group being optionally substituted, one or more times, identically or differently, with a substituent selected from: halo-, hydroxy-, cyano-, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, C₁-C₆-alkoxy-C₁-C₆-alkyl-, halo-C₁-C₆-alkoxy-C₁-C₆-alkyl-, R⁸—(C₁-C₆-alkyl)-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(W)C(═O)R⁸, —N(H)C(═O)NR⁸R⁷, —N(R⁷)C(═O)NR⁸R⁷, —NR⁸R⁷, —C(═O)N(H)R⁸, —C(═O)NR⁸R⁷, R⁸—S—, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group; wherein said C₁-C₆-alkyl- or C₃-C₆-cycloalkyl- group is optionally substituted, one or more times, identically or differently, with a substituent selected from:
   halo-, hydroxy-, —NHR⁷, —NR⁷R⁷, —N(C₁-C₃-alkyl)-C(═O)R⁷, —N(C₁-C₃-alkyl)-C(═O)OR⁷, C₁-C₃-alkyl-, R⁷—S(═O)₂—, C₁-C₃-alkoxy-, halo-C₁-C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, p,
   represent, independently from each other, an integer of 0, 1, 2 or 3;
and
q represents an integer of 0, 1, 2 or 3;
   or a tautomer, an N-oxide, a hydrate, a solvate, or a salt thereof, or a mixture of same.

In another preferred embodiment, the invention relates to compounds of formula (I):

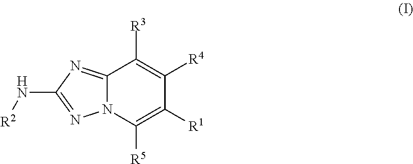

in which:
R¹ represents

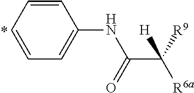

wherein * indicates the point of attachment of said group with the rest of the molecule;
R² represents a group selected from:

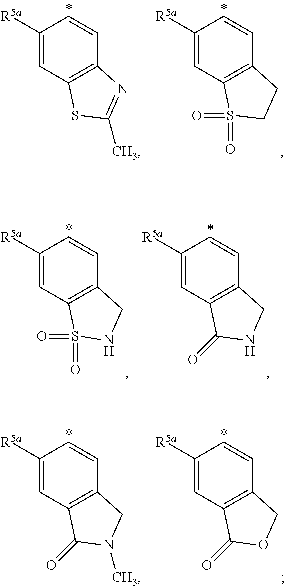

wherein * indicates the point of attachment of said groups with the rest of the molecule;
R³ represents a hydrogen atom;
R⁴ represents a hydrogen atom;
R⁵ represents a hydrogen atom;
R^{5a} represents a group selected from:
   F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃CH₂—O—;
R^{6a} represents a phenyl- group which is optionally substituted, one or more times, identically or differently, with a substituent selected from: halo-, methyl-, methoxy-;
R⁹ represents a group selected from:
   C₁-C₃-alkyl-, hydroxy-C₁-C₃-alkyl-, —N(R¹⁰)R¹⁰, —C₁-C₂-alkyl-N(R¹⁰)R¹⁰;
R¹⁰ represents a hydrogen atom or a methyl- group;
   or a tautomer, an N-oxide, a hydrate, a solvate, or a salt thereof, or a mixture of same.

In another preferred embodiment, the invention relates to compounds of formula (I):

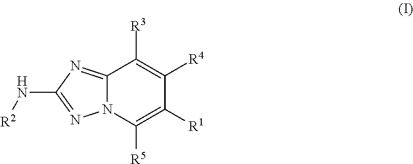

in which:
R¹ represents

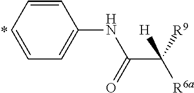

wherein * indicates the point of attachment of said group with the rest of the molecule;
R² represents a group selected from:

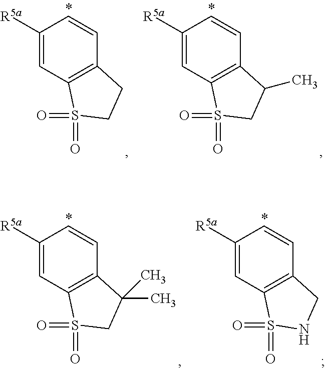

wherein * indicates the point of attachment of said groups with the rest of the molecule;
R³ represents a hydrogen atom;
R⁴ represents a hydrogen atom;
R⁵ represents a hydrogen atom;
R^{5a} represents a group selected from:
   F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃CH₂—O—;
R^{6a} represents a phenyl- group which is optionally substituted, one or more times, identically or differently, with a substituent selected from: halo-, methyl-, methoxy-;
R⁹ represents a group selected from:
   C₁-C₃-alkyl-, hydroxy-C₁-C₃-alkyl-, —N(R¹⁰)R¹⁰, —C₁-C₂-alkyl-N(R¹⁰)R¹⁰;
R¹⁰ represents a hydrogen atom or a methyl- group;
   or a tautomer, an N-oxide, a hydrate, a solvate, or a salt thereof, or a mixture of same.

In another preferred embodiment, the invention relates to compounds of formula (I):

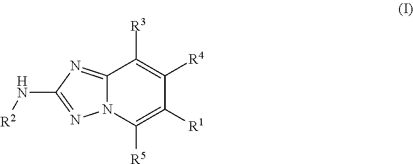

in which:
R¹ represents

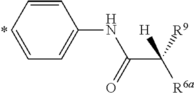

wherein * indicates the point of attachment of said group with the rest of the molecule;
R² represents:

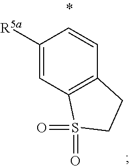

wherein * indicates the point of attachment of said groups with the rest of the molecule;
R³ represents a hydrogen atom;
R⁴ represents a hydrogen atom;
R⁵ represents a hydrogen atom;
R^{5a} represents a group selected from:
   F-, methyl-, methoxy-, ethoxy-, n-propoxy-, iso-propoxy-, cyclopropyl-O—, cyclopropyl-CH₂—O—, CH₃—O—CH₂CH₂—O—, CHF₂—O—, CF₃—O—, CF₃CH₂—O—;
R^{6a} represents a phenyl- group which is optionally substituted, one or more times, identically or differently, with a substituent selected from: halo-, methyl-, methoxy-;
R⁹ represents a group selected from:
   C₁-C₃-alkyl-, hydroxy-C₁-C₃-alkyl-, —N(R¹⁰)R¹⁰, —C₁-C₂-alkyl-N(R¹⁰)R¹⁰;
R¹⁰ represents a hydrogen atom or a methyl- group;
   or a tautomer, an N-oxide, a hydrate, a solvate, or a salt thereof, or a mixture of same.

More particularly still, the present invention covers compounds of general formula (I) which are disclosed in the Example section of this text, infra.

In accordance with another aspect, the present invention covers methods of preparing compounds of the present invention, said methods comprising the steps as described in the Experimental Section herein.

In a preferred embodiment, the present invention relates to a method of preparing compounds of general formula (I), supra, in which method an intermediate compound of general formula (5):

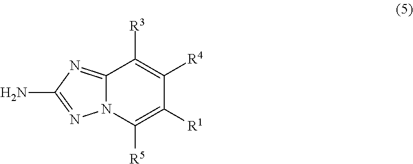

in which R¹, R³, R⁴, and R⁵ are as defined for the compounds of general formula (I), supra,
is allowed to react with an aryl compound of general formula (5a):

R²—Y (5a)

in which R² is as defined for the compounds of general formula (I), supra, and Y represents a leaving group, such as a halogen atom or a trifluoromethylsulphonyloxy or nonafluorobutylsulphonyloxy group for example,
thus providing a compound of general formula (I):

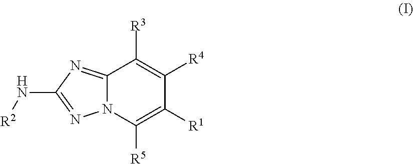

in which R¹, R², R³, R⁴, and R⁵ are as defined for the compounds of general formula (I), supra.

In another preferred embodiment, the present invention relates to a method of preparing compounds of general formula (I), supra, in which method an intermediate compound of general formula (7):

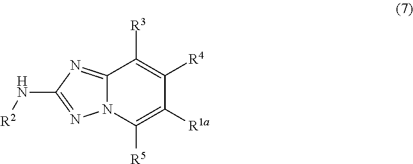

in which R², R³, R⁴, and R⁵ are as defined for the compounds of general formula (I), supra, and R^{1a} is a phenyl group to which an —NH₂ substituent is bound in the para position,
is allowed to react with a compound of general formula (7a):

R^{1b}—X (7a)

wherein R^{1b}—X represents

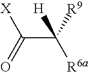

in which R⁹ and R^{6a} are as defined for the compounds of general formula (I), supra, and X is a suitable functional group (e.g. an —OH, —O—C₁-C₆-alkyl group, or a halogen atom), via which the R^{1b} of the R^{1b}—X compound (7a) can be coupled, via a coupling reaction, such as an amide coupling reaction for example, onto the —NH₂ substituent bound to the phenyl group R^{1a} of compound (7),
thus providing a compound of general formula (I):

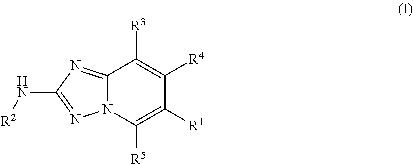

in which R¹, R², R³, R⁴, and R⁵ are as defined for the respective compounds of general formula (I), supra.

In another preferred embodiment, the present invention relates to a method of preparing compounds of general formula (I), supra, in which method an intermediate compound of general formula (7):

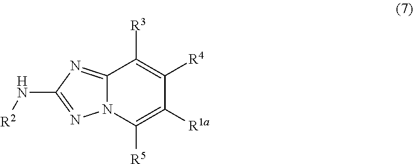

in which R², R³, R⁴, and R⁵ are as defined for the compounds of general formula (I), supra, and R^{1a} is a phenyl group to which an —NH₂ substituent is bound in the para position,
is allowed to react with a compound of general formula (7a):

R^{1b}—X (7a)

wherein R^{1b}—X represents

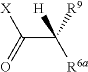

in which R⁹ and R^{6a} are as defined for the compounds of general formula (I), supra, and X is a suitable functional group (e.g. an —OH), via which the R^{1b} of the R^{1b}—X compound (7a) can be coupled, via a coupling reaction, such as an amide coupling reaction using a coupling reagent like for example HATU, and a base like for example sodium bicarbonate in an inert solvent like for example THF, DMF, DCM, NMP or mixtures thereof, onto the —NH₂ substituent bound to the phenyl group R^{1a} of compound (7),
thus providing a compound of general formula (I):

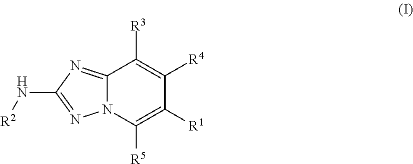

in which R¹, R², R³, R⁴, and R⁵ are as defined for the respective compounds of general formula (I), supra.

In another embodiment, the present invention relates to a method of preparing compounds of general formula (I), supra, in which method an intermediate compound of general formula (4):

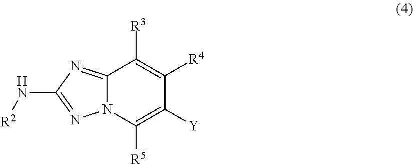

in which R², R³, R⁴, and R⁵ are as defined for the compound of general formula (I), supra, and Y represents a leaving group, such as a halogen atom or a trifluoromethylsulphonyloxy or nonafluorobutylsulphonyloxy group for example, is allowed to react with a compound of general formula (4a):

R¹—Z (4a)

in which R¹ is as defined for the compounds of general formula (I), supra, and Z represents a suitable functional group like for example a boronic acid or a boronic ester,
thus providing a compound of general formula (I):

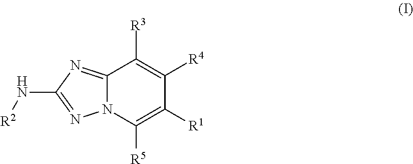

in which R¹, R², R³, R⁴, and R⁵ are as defined for the compounds of general formula (I), supra.

In accordance with a further aspect, the present invention covers intermediate compounds which are useful in the preparation of compounds of the present invention of general formula (I), particularly in the methods described herein.

### EXPERIMENTAL SECTION

The following Table lists the abbreviations used in this paragraph, and in the Examples section. NMR peak forms are stated as they appear in the spectra, possible higher order effects have not been considered.

| Abbreviation | Meaning |
|---|---|
| Ac | acetyl |
| BINAP | 2,2′-bis(diphenylphosphino)-1,1′-binaphthyl |
| Boc | tert-butyloxycarbonyl |
| br | broad |
| Brett-Phos | 2-(dicyclohexylphosphino)-3,6-dimethoxy-2′-4′-6′-tri-i-propyl-1,1′-biphenyl |
| c- | cyclo- |
| d | doublet |
| dd | doublet of doublets |
| DCM | dichloromethane |
| DME | 1,2-dimethoxyethane |
| DIPE | diisopropylether |
| DIPEA | N,N-diisopropylethylamine |
| DMF | N,N-dimethylformamide |
| DMSO | dimethyl sulfoxide |
| Dppf | 1,1′-bis(diphenylphosphino)ferrocene |
| Eq | equivalent |
| ESI | electrospray ionisation |
| HATU | N-[(dimethylamino)(3H-[1,2,3]triazolo[4,5-b]pyridin-3-yloxy)-methylene]-N- |
| | methylmethanaminium hexafluorophosphate |
| Hünig Base | N,N-diisopropylethylamine |
| m | multiplet |
| m.p. | melting point in ° C. |
| MS | mass spectrometry |
| MW | molecular weight |
| NaOtBu | sodium tert-butoxide; sodium 2-methylpropan-2-olate |
| NMP | N-methylpyrrolidinone |
| NMR | nuclear magnetic resonance spectroscopy: chemical shifts (δ) are given in ppm. |
| PdCl₂(PPh₃)₂ | dichlorobis(triphenylphosphine)palladium(II) |
| Pd(dba)₂ | bis-(dibenzylideneacetone)palladium(0) complex |
| Pd₂(dba)₃ | tris-(dibenzylideneacetone)dipalladium(0) chloroform complex |
| Pd(dppf)Cl₂ | dichloro[1,1′-bis(diphenylphosphino)ferrocene]palladium(II) |
| Pd(dppf)Cl₂•CH₂Cl₂ | dichloro[1,1′-bis(diphenylphosphino)ferrocene]palladium(II) dichloromethane |
| | adduct |
| Pd-Brett-Phos-pre-cat | chloro[2-(dicyclohexylphosphino)-3,6-dimethoxy-2′-4′-6′-tri-iso-propyl-1,1′- |
| | biphenyl][2-(2-aminoethyl)phenyl]palladium(II) |
| Pd-tBu-X-Phos-pre-cat | chloro(2-di-tert-butylphosphino-2′,4′,6′-tri-isopropyl-1,1′-biphenyl)[2-(2- |
| | aminoethyl)phenyl] palladium(II), |
| Pd-X-Phos-pre-cat | chloro(2-dicyclohexylphosphino-2′,4′,6′-tri-isopropyl-1,1′-biphenyl)[2-(2- |
| | aminoethyl)phenyl] palladium(II) methyl-tert-butylether adduct |
| PPh₃ | triphenylphosphine |
| P(oTol)₃ | tri-o-tolylphosphine |
| q | quartet |
| quin | quintett |
| Rac | racemic |
| Rt | room temperature |
| r.t. | room temperature |
| RT | retention time in minutes |
| s | singlet |
| t | triplet |
| TBAF | tetrabutylammoniumfluoride |
| tBu-X-Phos | 2-di-tert-butylphosphino-2′,4′6′-tri-isopropyl-1,1′-biphenyl |
| TBDPS | tert-butyldiphenylsilyl |
| TBTU | N-[(1H-benzotriazol-1-yloxy)(dimethylamino)methylene]-N- |
| | methylmethanaminium tetrafluoroborate |
| TEA | triethylamine |
| TFA | trifluoroacetic acid |
| THF | tetrahydrofuran |
| TMS | trimethylsilyl |
| Ts | para toluenesulfonyl; (tosyl) |
| UPLC | ultra performance liquid chromatography |
| X-Phos | 2-dicyclohexylphosphino-2′,4′,6′-triisopropylbiphenyl |

The schemes and procedures described below illustrate general synthetic routes to the compounds of general formula (I) of the invention and are not intended to be limiting. It is clear to the person skilled in the art that the order of transformations as exemplified in the schemes can be modified in various ways. The order of transformations exemplified in the schemes is therefore not intended to be limiting. In addition, interconversion of any of the substituents, in particular of R¹ or R², can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

A first reaction scheme is outlined infra:

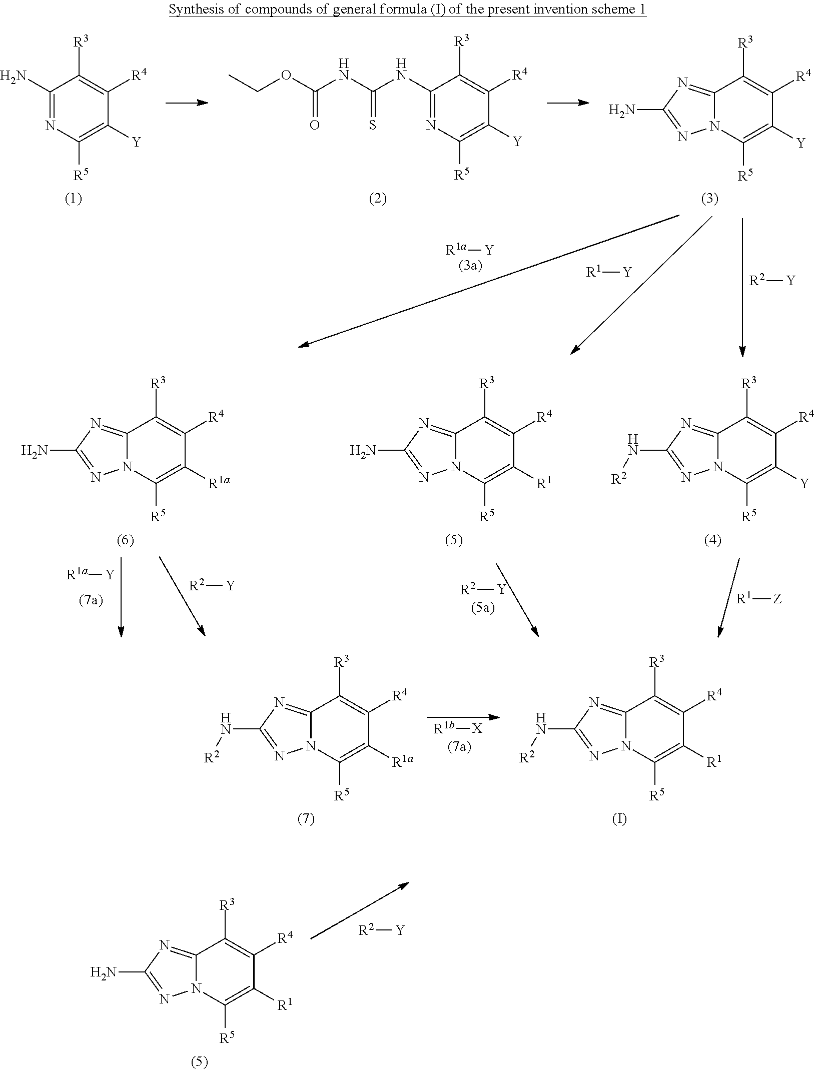

In scheme 1:

R¹, R², R³, R⁴, and R⁵ are as defined for the compounds of general formula (I), supra.

X represents a suitable functional group (e.g. an —OH or —O—C₁-C₆-alkyl group, or a halogen atom), via which the R^{1b} group of R^{1b}—X can be coupled, via a coupling reaction onto the respective functional group of R^{1a}, thus providing a compound of general formula (I). Examples of X, R^{1a}, and R^{1b} are given hereinafter.

Y represents a leaving group, such as a halogen atom or a trifluoromethylsulphonyloxy or nonafluorobutylsulphonyloxy group for example.

Z represents a suitable functional group via which the R¹ of the R¹—Z compound can be coupled, by a coupling reaction, onto the Y-bearing carbon atom of a compound (4), such as a halogen atom or a trifluoromethylsulphonyloxy or nonafluorobutylsulphonyloxy group for example, thereby replacing said Y with said R¹ moiety.

Compounds of general formula (I) can be synthesised according to the procedures depicted in scheme 1.

Many aryl halides of the formula R²—Y may be obtained commercially. Reagents of the general structure R^{1a}—Z and R¹—Z can for example be aryl boronic acids or aryl boronic esters. Many such reagents of the general structures R^{1a}—Z and R¹—Z are also commercially available. Reagents of the general structures R^{1a}—Z and R¹—Z can be prepared from aryl halides [see for example K. L. Billingslay, T. E. Barde, S. L Buchwald, Angew. Chem. 2007, 119, 5455 or T. Graening, Nachrichten aus der Chemie, January 2009, 57, 34].

The person skilled in the art will recognise that there are many precedented methods for synthesising suitable 3,4,6-substituted 5-halo-pyridin-2-ylamines of general formula (1); some 3,4,6-substituted 5-halo-pyridin-2-ylamines may be obtained commercially.

A suitably substituted 5-halo-pyridin-2-ylamine intermediate of general formula (1) is converted to the corresponding intermediate of general formula (2) by reaction with a suitable oxycarbonyl isothiocyanate, such as for example ethoxycarbonyl isothiocyanate at temperatures ranging from room temperature to the boiling point of the solvent, preferably room temperature [see for example M. Nettekoven, B. Püllmann, S. Schmitt, Synthesis 2003, 1643-1652].

Intermediates of general formula (2) may be converted to 6-Halo-[1,2,4]triazolo[1,5-a]pyridin-2-ylamine intermediates of general formula (3) by reaction with a suitable reagent, for example hydroxylammonium chloride, in presence of a suitable base, such as, for example DIPEA in a suitable solvent system, such as, for example, methanol, ethanol, 1-propanol, 2-propanol or mixtures of these solvents at elevated temperatures, e.g. 60° C. [see for example M. Nettekoven, B. Püllmann, S. Schmitt, Synthesis 2003, 1643-1652].

Intermediates of general formula (3) can be converted to intermediates of general formula (4) by reaction with suitable aryl compounds R²—Y, preferably aryl bromides, or aryl iodides or for example aryl trifluoromethylsulphonates or aryl nonafluorobutylsulphonates in the presence of a suitable base, such as, for example NaOtBu or cesium carbonate or potassium phosphate, and a suitable catalyst/ligand system, such as for example Pd₂(dba)₃/rac-BINAP, Pd₂dba₃/X-Phos, Pd₂dba₃/tBu-X-Phos, Pd₂dba₃/Brett-Phos, Pd-X-Phos-pre-cat/X-Phos, Pd-tBu-X-Phos-pre-cat/tBu-X-Phos, Pd-Brett-Phos-pre-cat/Brett-Phos in a suitable solvent such as THF, toluene, xylene, DME, or NMP, or mixtures of these solvents at temperatures ranging from room temperature to 200° C. The person skilled in the art will recognise that the appropriate choice of reaction conditions, such as temperature, choice of solvent and catalyst system is critical for preferred derivatization at the amino group of intermediates of general formula (3).

Intermediates of general formula (4) can be converted to compounds of general formula (I) by reaction with a suitable reagent R¹—Z, like for example a boronic acid derivative in the presence of a suitable catalyst system, like for example Pd(OAc)₂ and P(oTol)₃, or PdCl₂(PPh₃)₂ and PPh₃ and a suitable base, like for example aqueous potassium carbonate in a suitable solvent, like for example THF, DME, ethanol or 1-propanol or mixtures of these solvents at temperatures ranging from room temperature to 200° C., preferably the boiling point of the used solvent.

In an alternative route for the synthesis of compounds of general formula (I), intermediates of general formula (3) can be reacted with a suitable reagent R¹—Z, like for example a boronic acid derivative in the presence of a suitable catalyst system, like for example Pd(OAc)₂ and P(oTol)₃, or PdCl₂(PPh₃)₂ and PPh₃ and a suitable base, like for example aqueous potassium carbonate in a suitable solvent, like for example THF, DME, ethanol or 1-propanol or mixtures of these solvents at temperatures ranging from room temperature to 200° C., preferably the boiling point of the used solvent to furnish intermediates of the general formula (5).

Intermediates of general formula (5) can be converted to compounds of general formula (I) by reaction with suitable aryl compounds R²—Y, preferably aryl bromides, or aryl iodides or for example aryl trifluoromethylsulphonates or aryl nonafluorobutylsulphonates in the presence of a suitable base, such as, for example NaOtBu or cesium carbonate or potassium phosphate, and a suitable catalyst/ligand system, such as for example Pd₂(dba)₃/rac-BINAP, Pd₂dba₃/X-Phos, Pd₂dba₃/tBu-X-Phos, Pd₂dba₃/Brett-Phos, Pd-X-Phos-pre-cat/X-Phos, Pd-tBu-X-Phos-pre-cat/tBu-X-Phos, Pd-Brett-Phos-pre-cat/Brett-Phos in a suitable solvent such as THF, toluene, xylene, DME, or NMP, or mixtures of these solvents at temperatures ranging from room temperature to the 200° C.

Also as depicted in scheme 1, is a further alternative route for the synthesis of compounds of general formula (I): Intermediates of general formula (3) can be converted to intermediates of general formula (6) by a coupling reaction with a reagent R^{1a}—Z as described supra for synthesis of intermediate of general formula (5), thereby replacing said Y of intermediates of general formula (3) with said R^{1a} moiety.

Intermediates of general formula (6) can then be converted to intermediates of general formula (7) by a coupling reaction with a reagent R²—Y as described supra for synthesis of intermediates of general formula (4), thereby forming a bond between NH and said R² moiety.

Intermediates of general formula (7) can then be converted to compounds of general formula (I) by one or more further transformations. These can be modifications such as cleavage of protecting groups, reduction or oxidation of functional groups, halogenation, metallation, substitution or other reactions known to the person skilled in the art, for example the formation of an amide bond, thereby converting R^{1a} to said R¹ moiety.

Also as depicted in Scheme 1 is a further alternative route for the synthesis of compounds of general formula (I): Intermediates of general formula (3) can be converted to intermediates of general formula (6) by a coupling reaction with a reagent R^{1a}—Z as described supra for synthesis of intermediate of general formula (5), thereby replacing said Y of intermediates of general formula (3) with said R^{1a} moiety.

Intermediates of general formula (6) can then be converted to intermediates of general formula (5) by one or more further transformations. These can be modifications such as cleavage of protecting groups, reduction or oxidation of functional groups, halogenation, metallation, substitution or other reactions known to the person skilled in the art, for example the formation of an amide bond, thereby converting R^{1a} to said R¹ moiety.

Intermediates of general formula (5) can then be converted to compounds of general formula (I) by a coupling reaction with a reagent R²—Y as described supra for synthesis of intermediates of general formula (4), thereby forming a bond between NH and said R² moiety.

Each of the Schemes 2-3, infra, illustrates specific transformations for the synthesis of some selected compounds according to general formula (I).

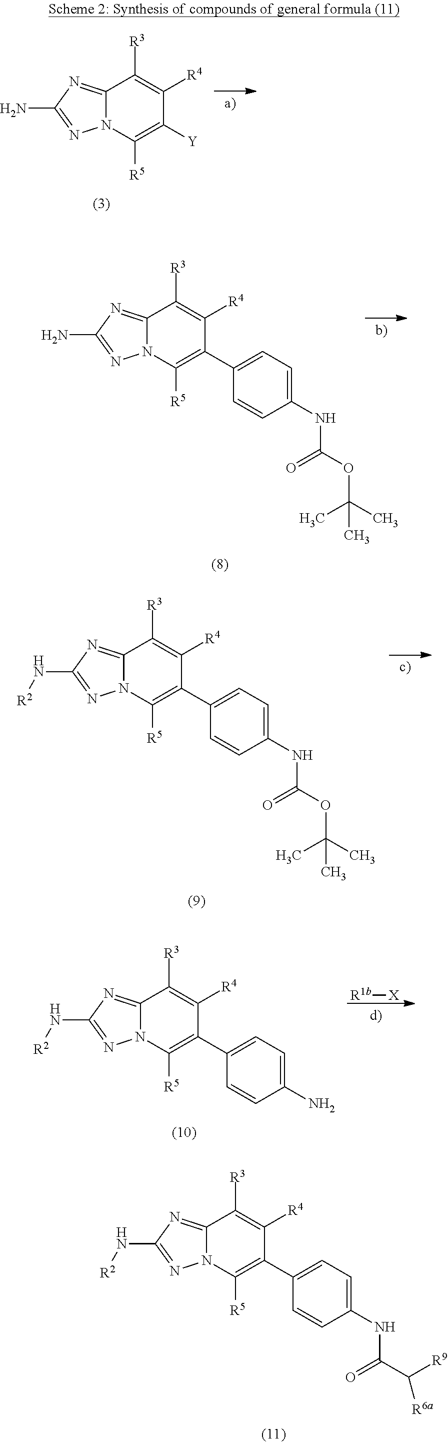

Scheme 2: Synthesis of compounds of general formula (11), wherein R², R³, R⁴, R⁵ and R^{6a} are as defined for the compounds of general formula (I), supra. Y is a leaving group, e.g. a halogen.

R⁹ represents a group selected from: C₁-C₃-alkyl-, hydroxy-C₁-C₃-alkyl-, —N(H)R⁸, —N(R⁷)R⁸, N(H)(R⁸)—C₁-C₃-alkyl-, N(R⁷)(R⁸)—C₁-C₃-alkyl-, PG′-O—C₁-C₃-alkyl-, —N(PG²)R⁸, N(PG²)(R⁸)—C₁-C₃-alkyl-.

a) coupling reaction using conditions as described herein for synthesis of intermediates of general formula (6);

b) coupling reaction using conditions as described herein for synthesis of intermediates of general formula (7);

c) removal of a Boc-protecting group using conditions known to the person skilled in the art (see for example T. W. Greene and P. G. M. Wuts in Protective Groups in Organic Synthesis, 3^{rd} edition, Wiley 1999);

d) conditions for the formation of an amide bond, e.g. using coupling reagents like for example HATU or TBTU and a base like for example potassium carbonate, sodium bicarbonate or DIPEA in an inert solvent like for example THF, DMF, DCM, NMP or mixtures thereof. Optionally, the removal of a protecting group is included in step d) if R⁹ represents PG′-O—C₁-C₃-alkyl-, —N(PG²)R⁸, or N(PG²)(R⁸)—C₁-C₃-alkyl- (see for example T. W. Greene and P. G. M. Wuts in Protective Groups in Organic Synthesis, 3^{rd} edition, Wiley 1999).

Preferably, in step d) a chiral compound of formula 7a:

R^{1b}—X (7a)

in which R^{1b} represents

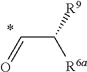

wherein * indicates the point of attachment of said group with the rest of the molecule; R⁹ represents a group selected from: C₁-C₃-alkyl-, hydroxy-C₁-C₃-alkyl-, —N(H)R⁸; —N(R⁷)R⁸, N(H)(R⁸)—C₁-C₃-alkyl-, N(R⁷)(R⁸)—C₁-C₃-alkyl-, PG′-O—C₁-C₃-alkyl-, —N(PG²)R⁸, N(PG²)(R⁸)—C₁-C₃-alkyl-; and
R^{6a}, R⁷ and R⁸ are as defined for the compounds of general formula (I), supra, and

X represents a suitable functional group (e.g. an —OH or —O—C₁-C₆-alkyl group, or a halogen atom), via which the R^{1b} group of R^{1b}—X can be coupled, via a coupling reaction onto the —NH₂ substituent bound to the phenyl group of R^{1a}, thus providing a compound of general formula (I), supra, is used for the formation of the amide bond.

Otherwise, a separation step may be required in order to separate the desired chiral compound of formula (I) from its respective antipode.

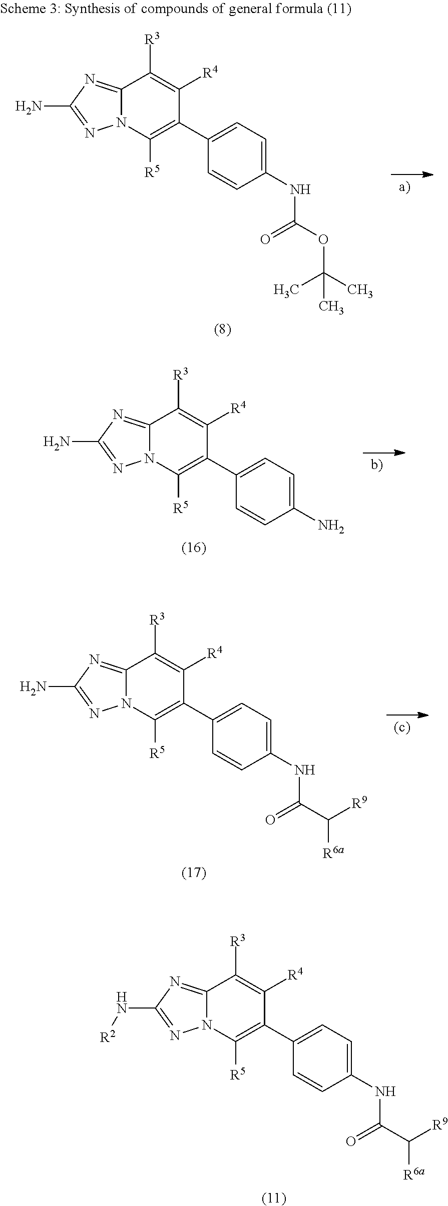

Scheme 3: Synthesis of compounds of general formula (11), wherein

R⁹ represents a group selected from: C₁-C₃-alkyl-, hydroxy-C₁-C₃-alkyl-, —N(H)R⁸, —N(R⁷)R⁸, N(H)(R⁸)—C₁-C₃-alkyl-, N(R⁷)(R⁸)—C₁-C₃-alkyl-, PG′-O—C₁-C₃-alkyl-, —N(PG²)R⁸, N(PG²)(R⁸)—C₁-C₃-alkyl-; and

R², R³, R⁴, R⁵, R^{6a}, R⁷, and R⁸ are as defined for the compounds of general formula (I), supra.

a) removal of a Boc-protecting group using conditions known to the person skilled in the art (see for example T. W. Greene and P. G. M. Wuts in Protective Groups in Organic Synthesis, 3^{rd} edition, Wiley 1999);

b) conditions for the formation of an amide bond, e.g. using coupling reagents like for example HATU or TBTU and a base like for example potassium carbonate, sodium bicarbonate or DIPEA in an inert solvent like for example THF, DMF, DCM, NMP or mixtures thereof;

c) coupling reaction using conditions as described supra for synthesis of intermediates of general formula (4). Optionally, the removal of a protecting group is included in step c) if R⁹ represents PG′-O—C₁-C₃-alkyl-, —N(PG²)R⁸, or N(PG²)(R⁸)—C₁-C₃-alkyl- (see for example T. W. Greene and P. G. M. Wuts in Protective Groups in Organic Synthesis, 3^{rd} edition, Wiley 1999).

Preferably, steps b) and c) are performed with achiral compounds and a separation of the desired chiral compound of formula (I) from its respective antipode is conducted after the coupling reaction according to step c).

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallisation. In some cases, impurities may be stirred out using a suitable solvent. In some cases, the compounds may be purified by chromatography, particularly flash chromatography, using for example pre-packed silica gel cartridges, e.g. from Separtis such as Isolute® Flash silica gel (silica gel chromatography) or Isolute® Flash NH₂ silica gel (aminophase-silica-gel chromatography) in combination with a suitable chromatographic system such as a Flashmaster II (Separtis) or an Isolera system (Biotage) and eluents such as, for example, gradients of hexane/ethyl acetate or DCM/methanol. In some cases, the compounds may be purified by preparative HPLC using, for example, a Waters autopurifier equipped with a diode array detector and/or on-line electrospray ionisation mass spectrometer in combination with a suitable pre-packed reverse phase column and eluants such as, for example, gradients of water and acetonitrile which may contain additives such as trifluoroacetic acid, formic acid or aqueous ammonia.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Nat”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

Analytical UPLC-MS was performed as follows:

Method A: System: UPLC Acquity (Waters) with PDA Detector and Waters ZQ mass spectrometer; Column: Acquity BEH C18 1.7 μm 2.1×50 mm; Temperature: 60° C.; Solvent A: Water+0.1% formic acid; Solvent B: acetonitrile; Gradient: 99% A→1% A (1.6 min)→1% A (0.4 min); Flow: 0.8 mL/min; Injection Volume: 1.0 μl (0.1 mg-1 mg/mL sample concentration); Detection: PDA scan range 210-400 nm—Fixed and ESI (+), scan range 170-800 m/z

Synthesis of Intermediate Compounds

### Intermediate Example Int01.01

### Ethyl [(5-bromopyridin-2-yl)carbamothioyl]carbamate

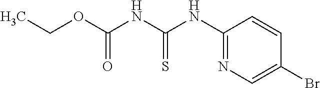 [SMILES: CCOC(=O)NC(=S)Nc1ccc(Br)cn1]

Ethoxycarbonyl isothiocyanate (16.7 g) was added to a stirred solution of 2-amino-5-brompyridine (20 g) in dioxane (200 mL). The mixture was stirred for 2 h at r.t. A white solid precipitated. Hexane (20 mL) was added and the white solid was collected by filtration.

Yield: 30.4 g of the title compound.

¹H-NMR (300 MHz, DMSO-d₆): δ [ppm]=1.22 (t, 3H), 4.19 (q, 2H), 8.08 (dd, 1H), 8.49 (d, 1H), 8.57 (br. d, 1H), 11.37-12.35 (m, 2H).

### Intermediate Example Int01.02

### 6-Bromo[1,2,4]triazolo[1,5-a]pyridin-2-amine

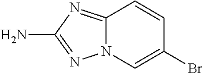 [SMILES: Nc1nc2ccc(Br)cn2n1]

Hydroxylammonium chloride (39.8 g) was suspended in methanol (200 mL), and ethanol (190 mL) and Hünig Base (59 mL) were added at r.t. The mixture was heated to 60° C., Int01.01 (30 g) was added portionwise, and the mixture was stirred at 60° C. for 2 h. The solvent was removed in vacuum, and water (150 mL) was added. A solid was collected by filtration and was washed with water and dried in vacuum.

Yield: 19.3 g of the title compound.

¹H-NMR (300 MHz, DMSO-d₆): δ [ppm]=6.10 (s, 2H), 7.28 (dd, 1H), 7.51 (dd, 1H), 8.88 (dd, 1H).

### Intermediate Example Int01.03

### tert-butyl [4-(2-amino[1,2,4]triazolo[1,5-a]pyridin-6-yl)phenyl]carbamate

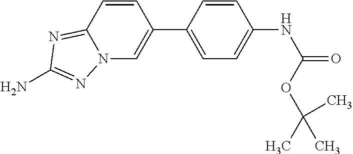 [SMILES: CC(C)(C)OC(=O)Nc1ccc(-c2ccc3nc(N)nn3c2)cc1]

To a stirred solution of Int01.02 (5.82 g) in 1-propanol (400 mL) was added 2M potassium carbonate solution (41 mL), {4-[(tert-butoxycarbonyl) amino] phenyl} boronic acid (8.6 g), triphenylphosphine (150 mg) and PdCl₂(PPh₃)₂ (1.9 g). The mixture was heated to reflux for 4 h, the solvent was removed in vacuum, water (150 mL) was added and the mixture was extracted with ethyl acetate (500 mL). The organic phase was dried (sodium sulfate), filtered through Celite and the solvent was removed in vacuum. The residue was triturated with DCM to give the title compound as a white solid. Yield: 7.2 g. ¹H-NMR (400 MHz, DMSO-d₆): δ [ppm]=1.37-1.55 (m, 9H), 5.99 (s, 2H), 7.36 (dd, 1H), 7.48-7.55 (m, 2H), 7.55-7.62 (m, 2H), 7.69 (dd, 1H), 8.78 (dd, 1H), 9.44 (s, 1H).

### Intermediate Example Int01.04

### 6-(4-aminophenyl)[1,2,4]triazolo[1,5-a]pyridin-2-amine

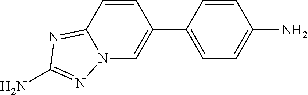 [SMILES: Nc1ccc(-c2ccc3nc(N)nn3c2)cc1]

To a stirred suspension of Int01.03 (7.05 g) in DCM (210 mL) was added TFA (66 mL). The mixture was stirred at r.t. for 1 h. The mixture was concentrated in vacuum. A saturated solution of potassium carbonate was added, until pH 10 was reached and the mixture was extracted for three times with DCM and methanol (10:1). The organic phase was dried (sodium sulfate) and the solvent was removed in vacuum to give 4.6 g of the title compound.

¹H-NMR (300 MHz, DMSO-d₆): δ [ppm]=5.26 (s, 2H), 5.95 (s, 2H), 6.64 (d, 2H), 7.29-7.45 (m, 3H), 7.64 (dd, 1H), 8.60-8.70 (m, 1H).

### Intermediate Example Int01.05

### N-[4-(2-amino[1,2,4]triazolo[1,5-a]pyridin-6-yl)phenyl]-2-(4-fluorophenyl)propanamide

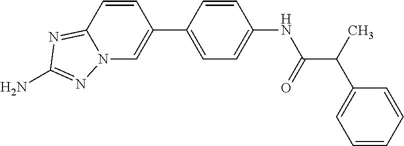 [SMILES: CC(C(=O)Nc1ccc(-c2ccc3nc(N)nn3c2)cc1)c1ccccc1]

To a stirred solution of Int01.04 (3.80 g) in DMF (350 mL) was added potassium carbonate (11.6 g), Int09.02 (5.67 g) and HATU (12.8 g). The mixture was stirred at room temperature for 2 h. Water was added, the mixture was stirred for 15 minutes and the mixture was extracted with ethyl acetate. The organic phase was washed with saturated sodium chloride solution, dried (sodium sulfate) and the solvent was removed in vacuum. The crude product was triturated with ethyl acetate to give 4.07 g of the title compound.

¹H-NMR (400 MHz, DMSO-d₆): δ [ppm]=1.39 (d, 3H), 3.83 (q, 1H), 5.98 (s, 2H), 7.08-7.17 (m, 2H), 7.32-7.44 (m, 3H), 7.60-7.67 (m, 4H), 7.70 (dd, 1H), 8.79 (d, 1H), 10.13 (s, 1H).

### Intermediate Example Int02.01

### methyl 4-bromo-3-methoxybenzoate

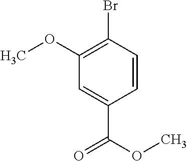

To a stirred solution of methyl 4-bromo-3-hydroxybenzoate (10.0 g) in DMF (50 mL) was added potassium carbonate (17.9 g) and iodomethane (9.2 g). The mixture was stirred at room temperature for 2 h. Ethyl acetate was added and the mixture was washed with water. The organic phase was washed with saturated sodium chloride solution, dried (sodium sulfate) and the solvent was removed in vacuum to give 10 g of the title compound, that was used without further purification.

¹H-NMR (400 MHz, DMSO-d₆): δ [ppm]=3.82 (s, 3H), 3.87 (s, 3H), 7.41 (dd, 1H), 7.47 (d, 1H), 7.67 (d, 1H).

### Intermediate Example Int02.02

### 4-bromo-3-methoxybenzoic acid

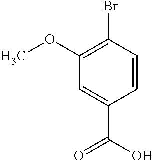

To a stirred solution of methyl 4-bromo-3-methoxybenzoate (11.2 g) in THF (130 mL), methanol (45 mL) and water (45 mL) was added a 1 M solution of lithium hydroxide in water (140 mL). The mixture was stirred at room temperature for 1 h. The solvent was removed in vacuum. Water was added and 1 N hydrochloric acid was added with ice bath cooling until pH 4 was reached. The precipitated solid was collected by filtration, washed with water and dried in vacuum to give 10.1 g of the title compound, that was used without further purification.

¹H-NMR (300 MHz, DMSO-d₆): δ [ppm]=3.87 (s, 3H), 7.42 (dd, 1H), 7.50 (d, 1H), 7.68 (d, 1H), 13.21 (br. s., 1H).

### Intermediate Example Int02.03

### 4-bromo-3-methoxy-N-(2,2,2-trifluoroethyl)benzamide

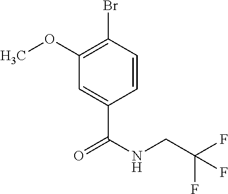 [SMILES: COc1cc(C(=O)NCC(F)(F)F)ccc1Br]

To a stirred suspension of 4-bromo-3-methoxybenzoic acid (2.0 g) in THF (100 mL) was added 2,2,2-trifluoroethylamine (1.26 g), HATU (3.87 g), and DIPEA (1.7 mL). The mixture was stirred at room temperature for 12 h. Water (350 ml) and saturated sodium bicarbonate solution (350 ml) were added. The organic phase was separated and the aqueous phase was extracted with ethyl acetate. The combined organic extracts were dried (sodium sulfate) and the solvent was removed in vacuum. Silica gel chromatography gave 2.57 g of the title compound.

¹H-NMR (400 MHz, DMSO-d₆): δ [ppm]=3.92 (s, 3H), 4.11 (qd, 2H), 7.43 (dd, 1H), 7.56 (d, 1H), 7.72 (d, 1H), 9.19 (t, 1H).

### Intermediate Example Int02.06

### 4-bromo-3-methoxy-N, N-dimethylbenzamide

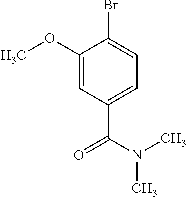

Starting from 4-bromo-3-methoxybenzoic acid and dimethyl amine, Int02.06 was prepared analogously to the procedure for the preparation of Int02.05.

### Intermediate Example Int03.01

### 1-bromo-2-methoxy-4-(methylsulfanyl)benzene

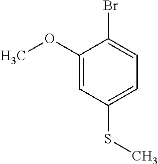

To a stirred solution of 1-bromo-4-fluoro-2-methoxybenzene (4.0 g) in DMF (40 mL) was added sodium methanethiolate (2.76 g). The mixture was stirred at room temperature for 30 minutes and at 85° C. for 2 h. Water was added and the mixture was extracted with ethyl acetate. The organic phase was washed with saturated sodium chloride solution, dried (sodium sulfate) and the solvent was removed in vacuum. Silica gel chromatography gave 280 mg of the title compound.

¹H-NMR (400 MHz, DMSO-d₆): δ [ppm]=2.46 (s, 3H), 3.82 (s, 3H), 6.74 (dd, 1H), 6.91 (d, 1H), 7.44 (d, 1H).

### 1-bromo-2-methoxy-4-(methylsulfanyl)benzene (alternative protocol)

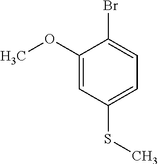

To a stirred solution of 1-bromo-4-fluoro-2-methoxybenzene (10.0 g) in DMF (100 mL) was added sodium methanethiolate (4.44 g). The mixture was stirred at 65° C. for 2 h. The mixture was cooled to 0° C. and methyl iodide (4.55 mL) was added. The mixture was stirred at room temperature for 1 h and further sodium methanethiolate (4.44 g) was added. The mixture was stirred at 65° C. for 1 h. The mixture was cooled to 0° C. and methyl iodide (4.55 mL) was added. The mixture was stirred at room temperature for 1 h. Water was added and the mixture was extracted with ethyl acetate. The organic phase was washed with saturated sodium chloride solution, dried (sodium sulfate) and the solvent was removed in vacuum. Silica gel chromatography gave 6.2 g of the title compound as a 2:1 mixture with the starting material. The mixture was used for the next step without purification.

### Intermediate Example Int03.02

### 1-bromo-2-methoxy-4-(methylsulfonyl)benzene

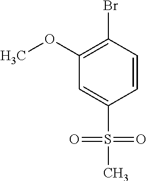

To a stirred solution of Int03.01 (265 mg) in chloroform (10 mL) was added 3-chlorobenzenecarboperoxoic acid (mCPBA) (890 mg). The mixture was stirred at room temperature for 1 h. A half-saturated solution of sodium bicarbonate was added and the mixture was extracted with dichloromethane. The organic phase was washed with saturated sodium chloride solution, dried (sodium sulfate) and the solvent was removed in vacuum. Silica gel chromatography gave 252 mg of the title compound.

¹H-NMR (300 MHz, DMSO-d₆): δ [ppm]=3.22 (s, 3H), 3.93 (s, 3H), 7.39 (dd, 1H), 7.50 (d, 1H), 7.84 (d, 1H).

### Intermediate Example Int04.01

### 1-bromo-2-ethoxy-4-fluorobenzene

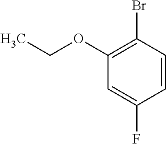

To a stirred solution of 2-bromo-5-fluorophenol (5.0 g) in DMF (30 mL) was added potassium carbonate (10.8 g) and iodoethane (6.12 g). The mixture was stirred at room temperature for 16 h. The solvent was removed in vacuum. Water was added and the mixture was extracted with a mixture of ethyl acetate and hexane (3:1). The organic phase was washed with saturated sodium chloride solution, dried (sodium sulfate) and the solvent was removed in vacuum, to give 5.06 g of the title compound as a crude product, that was used for the next step without purification.

¹H-NMR (400 MHz, DMSO-d₆): δ [ppm]=1.31 (t, 3H), 4.08 (q, 2H), 6.71 (td, 1H), 7.00 (dd, 1H), 7.55 (dd, 1H).

### Intermediate Example Int04.02

### 1-bromo-2-ethoxy-4-(methylsulfanyl)benzene

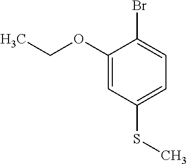

To a stirred solution of 1-bromo-2-ethoxy-4-fluorobenzene (2.0 g) in DMF (20 mL) was added sodium methanethiolate (1.66 g). The mixture was stirred for 2 h at 65° C. The mixture was cooled to room temperature and ethyl iodide (1.3 mL) was added. The mixture was stirred at room temperature for 1 h. Water was added and the mixture was extracted with ethyl acetate. The organic phase was washed with saturated sodium chloride solution, dried (sodium sulfate) and the solvent was removed in vacuum. Silica gel chromatography gave 1.65 g of the title compound.

¹H-NMR (400 MHz, DMSO-d₆): δ [ppm]=1.24-1.36 (m, 3H), 2.45 (s, 3H), 4.08 (q, 2H), 6.73 (dd, 1H), 6.89 (d, 1H), 7.43 (d, 1H).

### Intermediate Example Int04.03

### 1-bromo-2-ethoxy-4-(methylsulfonyl)benzene

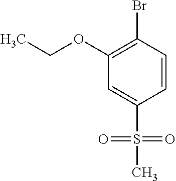

To a stirred solution of Int04.02 (1.65 g) in chloroform (65 mL) was added 3-chlorobenzenecarboperoxoic acid (mCPBA) (4.49 g). The mixture was stirred at room temperature for 16 h. With ice bath cooling, a half-saturated solution of sodium bicarbonate and a 0.2 M solution of sodium thiosulfate was added, the mixture was stirred for 30 minutes and the mixture was extracted with dichloromethane. The organic phase was washed with saturated sodium chloride solution, dried (sodium sulfate) and the solvent was removed in vacuum. Silica gel chromatography gave 1.35 g of the title compound.

¹H-NMR (300 MHz, DMSO-d₆): δ [ppm]=1.35 (t, 3H), 3.22 (s, 3H), 4.20 (q, 2H), 7.37 (dd, 1H), 7.48 (d, 1H), 7.84 (d, 1H).

### Intermediate Example Int05.01

### 1-bromo-4-fluoro-2-(2,2,2-trifluoroethoxy)benzene

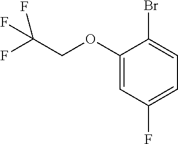 [SMILES: Fc1ccc(Br)c(OCC(F)(F)F)c1]

To a stirred solution of 2-bromo-5-fluorophenol (1.5 g) in acetonitrile (0.5 mL) and DMF (8.5 mL) in a microwave tube was added potassium carbonate (2.1 g) and 2,2,2-trifluoroethyl trifluoromethanesulfonate (2.37 g). The mixture was heated to 150° C. in a microwave oven for 30 minutes. In a second microwave tube the same reaction was repeated. Both mixtures were combined. The solvent was removed in vacuum, ethyl acetate and hexane (1:1) was added and the mixture was washed with water. The organic phase was washed with saturated sodium chloride solution, dried (sodium sulfate) and the solvent was removed in vacuum. Silica gel chromatography gave 4.0 g of the title compound.

¹H-NMR (300 MHz, CHLOROFORM-d): δ [ppm]=4.39 (q, 2H), 6.62-6.78 (m, 2H), 7.53 (dd, 1H).

### Intermediate Example Int05.02

### 1-bromo-4-(methylsulfanyl)-2-(2, 2, 2-trifluoroethoxy)benzene

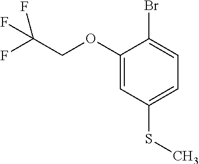

To a stirred solution of Int05.01 (4.0 g) in DMF (15 mL) was added sodium methanethiolate (1.0 g). The mixture was stirred for 2 h at 60° C. The mixture was cooled to room temperature. Water was added and the mixture was extracted with ethyl acetate. The organic phase was washed with saturated sodium chloride solution, dried (sodium sulfate) and the solvent was removed in vacuum to give 3.8 g of the crude title compound, that was used for the next step without purification.

¹H-NMR (300 MHz, CHLOROFORM-d): δ [ppm]=2.48 (s, 3H), 4.39 (q, 2H), 6.78-6.88 (m, 2H), 7.46 (d, 1H).

### Intermediate Example Int05.03

### 1-bromo-4-(methylsulfonyl)-2-(2,2,2-trifluoroethoxy)benzene

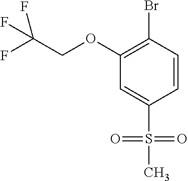 [SMILES: CS(=O)(=O)c1ccc(Br)c(OCC(F)(F)F)c1]

To a stirred solution of Int05.02 (3.8 g) in chloroform (100 mL) was added 3-chlorobenzenecarboperoxoic acid (mCPBA) (8.48 g). The mixture was stirred at room temperature for 16 h. With ice bath cooling, a half-saturated solution of sodium bicarbonate and a 0.2 M solution of sodium thiosulfate was added, the mixture was stirred for 30 minutes and the mixture was extracted with dichloromethane. The organic phase was washed with a 0.2 M solution of sodium thiosulfate and a saturated sodium chloride solution, dried (sodium sulfate) and the solvent was removed in vacuum. Silica gel chromatography gave a solid that was triturated with ether to give 2.1 g of the title compound.

¹H-NMR (400 MHz, CHLOROFORM-d): δ [ppm]=3.06 (s, 3H), 4.50 (q, 2H), 7.45 (d, 1H), 7.52 (dd, 1H), 7.81 (d, 1H).

### Intermediate Example Int06.01

### methyl 4-bromo-3-(2,2,2-trifluoroethoxy)benzoate

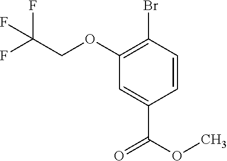 [SMILES: COC(=O)c1ccc(Br)c(OCC(F)(F)F)c1]

To a stirred solution of methyl 4-bromo-3-hydroxybenzoate (2.5 g) in acetonitrile (0.5 mL) and DMF (10 mL) in a microwave tube was added potassium carbonate (2.93 g) and 2,2,2-trifluoroethyl trifluoromethanesulfonate (2.79 g). The mixture was heated to 150° C. in a microwave oven for 30 minutes. The solvent was removed in vacuum, ethyl acetate was added and the mixture was washed with water. The organic phase was washed with saturated sodium chloride solution, dried (sodium sulfate) and the solvent was removed in vacuum. Recrystallization of the residue from ethanol gave 1.2 g of the title compound. The mother liquor was concentrated in vacuum and purified by aminophase-silica-gel chromatography followed by recrystallized from methanol and water to give further 0.64 g of the title compound.

¹H-NMR (300 MHz, CHLOROFORM-d): δ [ppm]=3.93 (s, 3H), 4.47 (q, 2H), 7.56 (d, 1H), 7.58-7.70 (m, 2H).

### Intermediate Example Int06.02

### 4-bromo-3-(2,2,2-trifluoroethoxy)benzoic acid

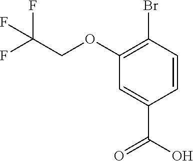 [SMILES: O=C(O)c1ccc(Br)c(OCC(F)(F)F)c1]

To a stirred solution of Int06.01 (1.83 g) in THF (30 mL), methanol (10 mL) and water (10 mL) was added a 1 M solution of lithium hydroxide in water (18 mL). The mixture was stirred at room temperature for 1 h. Water was added and 2 N hydrochloric acid was added until pH 4 was reached. The precipitated solid was collected by filtration, was washed with water. The solid was suspended with toluene and concentrated in vacuum. Trituration of the residue with hexane gave 1.6 g of the title compound.

¹H-NMR (300 MHz, DMSO-d₆): δ [ppm]=4.95 (q, 2H), 7.51 (dd, 1H), 7.65 (d, 1H), 7.74 (d, 1H), 13.29 (br. s., 1H).

### Intermediate Example Int06.03

### 4-bromo-3-(2, 2, 2-trifluoroethoxy)benzamide

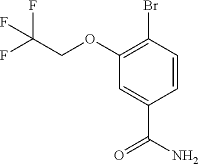 [SMILES: NC(=O)c1ccc(Br)c(OCC(F)(F)F)c1]

To a stirred suspension of Int06.02 (0.50 g) in THF (20 mL) was added DMF (0.2 mL) and oxalyl chloride (0.30 mL). The mixture was stirred at room temperature for 0.5 h. With ice bath cooling, ammonia gas was bubbled through the reaction mixture. A white solid precipitated. The mixture was stirred for further 15 minutes. Ethyl acetate was added and the mixture was washed with water and with a saturated solution of sodium chloride. The organic phase was dried (sodium sulfate) and the solvent was removed in vacuum to give a white solid. The solid was triturated with toluene and washed with toluene and hexanes to give 0.27 g of the title compound.

¹H-NMR (300 MHz, DMSO-d₆): δ [ppm]=4.88 (q, 2H), 7.45 (dd, 1H), 7.50 (br. s., 1H), 7.64 (d, 1H), 7.69 (d, 1H), 8.00 (br. s., 1H).

### Intermediate Example Int08.140

### methyl 2-(4-fluorophenyl)-3-hydroxypropanoate

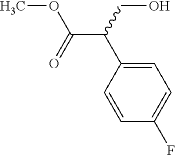

To a stirred solution of methyl (4-fluorophenyl)acetate (5.5 g) in DMSO (220 mL) was added 1,3,5-trioxane (3.24 g) and sodium methoxide (88 mg). The mixture was stirred at room temperature for 16 h. Water was added and the reaction mixture was extracted with ethyl acetate. The organic phase was washed with saturated sodium chloride solution, dried (sodium sulfate) and the solvent was removed in vacuum. Silica gel chromatography gave 3.8 g of the title compound.

¹H-NMR (400 MHz, DMSO-d₆): δ [ppm]=3.50-3.61 (m, 4H), 3.71-3.79 (m, 1H), 3.82-3.90 (m, 1H), 4.98 (t, 1H), 7.07-7.16 (m, 2H), 7.27-7.34 (m, 2H).

### Intermediate Example Int08.141 methyl 3-{[tert-butyl(diphenyl)silyl]oxy}-2-(4-fluorophenyl)propanoate

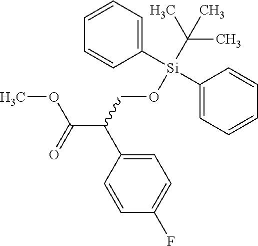 [SMILES: COC(=O)C(CO[Si](c1ccccc1)(c1ccccc1)C(C)(C)C)c1ccc(F)cc1]

To a stirred solution of imidazole (2.36 g) and tert-butyl(chloro)diphenylsilane (4.58 g) in DMF (90 mL) was added a solution of Int08.140 (2.75 g), dissolved in DMF (20 mL). The mixture was stirred at room temperature for 30 minutes. Water was added and the reaction mixture was extracted with ethyl acetate. The organic phase was washed with saturated sodium chloride solution, dried (sodium sulfate) and the solvent was removed in vacuum. Silica gel chromatography gave 5.3 g of the title compound.

¹H-NMR (300 MHz, DMSO-d₆): δ [ppm]=0.90 (s, 9H), 3.60 (s, 3H), 3.77 (dd, 1H), 3.92-4.00 (m, 1H), 4.02-4.11 (m, 1H), 7.05-7.16 (m, 2H), 7.24-7.33 (m, 2H), 7.33-7.46 (m, 6H), 7.46-7.57 (m, 4H).

### Intermediate Example Int08.142

### 3-{[tert-butyl(diphenyl)silyl]oxy}-2-(4-fluorophenyl)propanoic acid

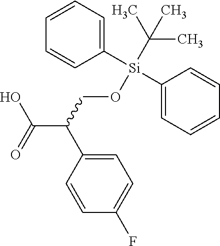 [SMILES: CC(C)(C)[Si](OCC(C(=O)O)c1ccc(F)cc1)(c1ccccc1)c1ccccc1]

To a stirred solution of Int08.141 (5.3 g) in 2-propanol (55 mL) was added a solution of sodium hydroxide (0.97 g), dissolved in water (18 mL). The mixture was stirred at 60° C. for 30 minutes. The solution was cooled to room temperature, saturated ammonium chloride solution was added and the reaction mixture was extracted with ethyl acetate. The organic phase was dried (sodium sulfate) and the solvent was removed in vacuum. Silica gel chromatography gave 5.3 g of the title compound.

¹H-NMR (300 MHz, DMSO-d₆): δ [ppm]=0.90 (s, 9H), 3.67-3.76 (m, 1H), 3.77-3.87 (m, 1H), 4.02-4.10 (m, 1H), 7.05-7.15 (m, 2H), 7.24-7.32 (m, 2H), 7.32-7.46 (m, 6H), 7.46-7.59 (m, 4H), 12.64 (br. s., 1H).

### Intermediate Example Int09.01

### Rac-methyl 2-(4-fluorophenyl)propanoate

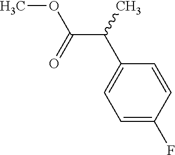

To a stirred solution of diisopropylamine (13.0 g) in tetrahydrofurane (160 mL) was added a solution of n-butyllithium in hexane (51.4 mL; c=2.5 M) at −78° C. The solution was stirred at 0° C. for 15 minutes. The solution was cooled to −78° C. and a solution of methyl (4-fluorophenyl)acetate (18.0 g), dissolved in tetrahydrofurane (40 mL) was added. The solution was stirred at −78° C. for 30 minutes. Methyl iodide (10.0 mL) was added at −78° C., and the solution was allowed to warm up to 0° C. within 1 h. Water was added and the reaction mixture was extracted with ethyl acetate. The organic phase was dried (sodium sulfate) and the solvent was removed in vacuum. Silica gel chromatography gave 18.9 g of the title compound.

¹H-NMR (400 MHz, DMSO-d₆): δ [ppm]=1.34 (d, 3H), 3.55 (s, 3H), 3.79 (q, 1H), 7.08-7.15 (m, 2H), 7.25-7.32 (m, 2H).

### Intermediate Example Int09.02

### Rac-2-(4-fluorophenyl)propanoic acid

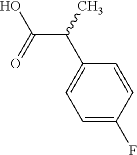

To a stirred solution of Int09.01 (18.9 g) in ethanol (200 mL) was added a solution of potassium hydroxide (35 g), dissolved in water (200 mL). The mixture was stirred at 0° C. for 4 h. Hydrochloric acid (c=4.0 M) was added until pH 5 was reached and the reaction mixture was extracted with ethyl acetate. The organic phase was separated and the solvent was removed in vacuum to give 15.64 g of the title product. The crude product was used without further purification.

¹H-NMR (300 MHz, DMSO-d₆): δ [ppm]=1.31 (d, 3H), 3.66 (q, 1H), 7.05-7.15 (m, 2H), 7.24-7.33 (m, 2H), 12.30 (s, 1H).

### Intermediate Example Int09.03

### (2R)-2-(4-fluorophenyl)propanoic acid

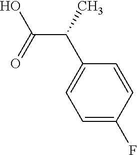

To a stirred solution of Int09.02 (23.6 g) in refluxing ethyl acetate (250 mL) was added a solution of (1S)-1-phenylethanamine (17.35 g) in ethyl acetate. The mixture was allowed to cool down to room temperature within 1 h. A white solid was collected by filtration, was washed with ethyl acetate and dried in vacuum to give 27.5 g of a solid. The solid was recrystallized from 400 mL refluxing ethyl acetate. The mixture was allowed to cool down to room temperature. A white solid was collected by filtration, was washed with ethyl acetate and dried in vacuum to give 18.3 g of a solid. The solid was twice recrystallized from refluxing ethyl acetate (350 mL; 300 mL). A white solid was collected by filtration, was washed with ethyl acetate and dried in vacuum to give 10.51 g of a solid. The solid was dissolved in water, hydrochloric acid (c=2.0 M) was added until pH 5 was reached and the reaction mixture was extracted with dichloromethane. The organic phase was dried (sodium sulfate) and the solvent was removed in vacuum to give 5.6 g of the title product. The crude product was used without further purification.

¹H-NMR (300 MHz, DMSO-d₆): δ [ppm]=1.31 (d, 3H), 3.66 (q, 1H), 7.05-7.16 (m, 2H), 7.24-7.33 (m, 2H), 12.28 (br. s., 1H).

[α]_{D}²⁰:79.3° (in DMSO)

Column: Chiralcel OJ-H 150×4.6; Flow: 1.00 mL/min; Solvent: A: Hexane, B: 2-propanol with 0.1% formic acid; Solvent mixture: 80% A+20% B. Run Time: 30 min. Retention Time: 3.41 min; UV 254 nm; Enantiomeric Ratio: 99.8%: 0.2%.

### Intermediate Example Int10.01

### 1-bromo-2-(cyclopropyloxy)-4-fluorobenzene

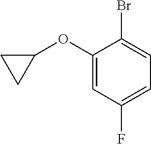 [SMILES: Fc1ccc(Br)c(OC2CC2)c1]

To a stirred solution of 2-bromo-5-fluorophenol (1.0 g) in DMF (15 mL) in a microwave tube was added cesium carbonate (5.0 g), potassium iodide (130 mg) and bromocyclopropane (1.82 g). The mixture was heated in a microwave oven to 180° C. for 1 h, to 200° C. for 1 h and to 220° C. for 1 h. Ethyl acetate was added and the mixture was washed with water. The organic phase was washed with saturated sodium chloride solution, dried (sodium sulfate) and the solvent was removed in vacuum. Silica gel chromatography gave 1.14 g of the title compound.

¹H-NMR (300 MHz, DMSO-d₆): δ [ppm]=0.62-0.88 (m, 4H), 3.90-4.00 (m, 1H), 6.77 (td, 1H), 7.23 (dd, 1H), 7.48-7.63 (m, 1H).

### Intermediate Example Int10.02

### 1-bromo-2-(cyclopropyloxy)-4-(methylsulfanyl)benzene

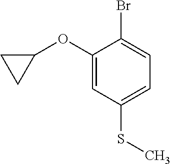

To a stirred solution of Int10.01 (1.4 g) in DMF (12 mL) was added sodium methanethiolate (546 mg). The mixture was for 2 h at 90° C. The mixture was cooled to room temperature, water was added and the mixture was extracted with ethyl acetate. The organic phase was washed with saturated sodium chloride solution, dried (sodium sulfate) and the solvent was removed in vacuum. Silica gel chromatography gave 1.17 g of the title compound.

¹H-NMR (300 MHz, DMSO-d₆): δ [ppm]=0.59-0.85 (m, 4H), 2.46 (s, 3H), 3.95 (tt, 1H), 6.77 (dd, 1H), 7.18 (d, 1H), 7.43 (d, 1H).

### Intermediate Example Int10.03

### 1-bromo-2-(cyclopropyloxy)-4-(methylsulfonyl)benzene

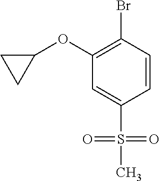

To a stirred solution of Int10.02 (1.15 g) in chloroform (45 mL) was added 3-chlorobenzenecarboperoxoic acid (mCPBA) (2.98 g). The mixture was stirred at room temperature for 2 h. With ice bath cooling, a half-saturated solution of sodium bicarbonate and a 0.2 M solution of sodium thiosulfate was added, the mixture was stirred for 30 minutes and the mixture was extracted with dichloromethane. The organic phase was washed with saturated sodium chloride solution, dried (sodium sulfate) and the solvent was removed in vacuum. Silica gel chromatography gave 0.91 g of the title compound.

¹H-NMR (300 MHz, DMSO-d₆): δ [ppm]=0.66-0.93 (m, 4H), 3.23 (s, 3H), 4.09 (tt, 1H), 7.43 (dd, 1H), 7.77 (d, 1H), 7.84 (d, 1H).

### Intermediate Example Intl 1.01

### Ethyl [(5-chloropyridin-2-yl)carbamothioyl]carbamate

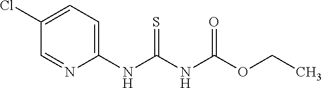 [SMILES: CCOC(=O)NC(=S)Nc1ccc(Cl)cn1]

Ethoxycarbonyl isothiocyanate (3.37 g) was added to a stirred solution of 2-amino-5-chloropyridine (3.0 g) in dioxane (100 mL). The mixture was stirred at r.t. for 14 h. The solvent was removed in vacuum. The solid was dissolved in dichloromethane and methanol (100:1), filtered and the solvent was removed in vacuum to give a solid that was recrystallized from ethyl acetate to give 4.4 g of the title compound.

¹H-NMR (400 MHz, CHLOROFORM-d): δ [ppm]=1.35 (3H), 4.31 (2H), 7.71 (1H), 8.03 (1H), 8.34 (1H), 8.83 (1H), 12.09 (1H).

### Intermediate Example Intl 1.02

### 6-chloro[1,2,4]triazolo[1,5-a]pyridin-2-amine

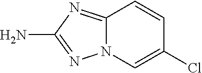 [SMILES: Nc1nc2ccc(Cl)cn2n1]

Hydroxylammonium chloride (4.4 g) was suspended in methanol (35 mL), and ethanol (35 mL) and Hünig Base (10.2 mL) were added at r.t. The mixture was heated to 60° C., Int11.01 (4.4 g) was added portionwise, and the mixture was stirred at 60° C. for 2 h. The solvent was removed in vacuum and water (150 mL) was added. A solid was collected by filtration and was washed with water and dried in vacuum.

Yield: 2.0 g of the title compound.

¹H-NMR (300 MHz, DMSO-d₆): δ [ppm]=6.09 (2H), 7.28-7.37 (1H), 7.39-7.49 (1H), 8.84 (1H).

### Intermediate Example Intl 1.03

### 6-chloro-N-[2-methoxy-4-(methylsulfonyl)phenyl][1,2,4]triazolo[1,5-a]pyridin-2-amine

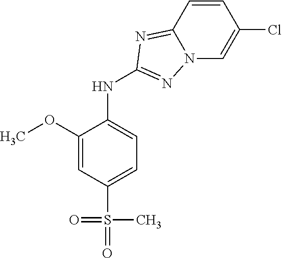 [SMILES: COc1cc(S(C)(=O)=O)ccc1Nc1nc2ccc(Cl)cn2n1]

To a stirred suspension of Int11.02 (0.7 g) in toluene (28 mL) was added Int03.02 (1.27 g), chloro(2-dicyclohexylphosphino-2′,4′,6′-tri-isopropyl-1,1′-biphenyl)[2-(2-aminoethyl)phenyl] palladium(II) methyl-tert-butylether adduct (343 mg), X-Phos (202 mg) and powdered potassium phosphate (3.09 g). The flask was degassed twice and backfilled with argon. The mixture was heated to reflux for 3 h. Further chloro(2-dicyclohexylphosphino-2′,4′,6′-tri-isopropyl-1,1′-biphenyl)[2-(2-aminoethyl)phenyl] palladium(II) methyl-tert-butylether adduct (30 mg) and X-Phos (19 mg) were added and the mixture was heated to reflux for 15 h. The solvent was removed in vacuum. Silica gel chromatography gave a solid that was triturated with ethyl acetate to give 1.0 g of the title compound.

¹H-NMR (400 MHz, DMSO-d₆): δ [ppm]=3.16 (3H), 3.95 (3H), 7.42 (1H), 7.50 (1H), 7.62-7.69 (2H), 8.41 (1H), 8.70 (1H), 9.17 (1H).

### Intermediate Example Intl 1.04

### (2R)-2-(4-fluorophenyl)-N-[4-(4,4,5,5-tetramethyl-1,3,2-dioxaborolan-2-yl)phenyl]propanamide

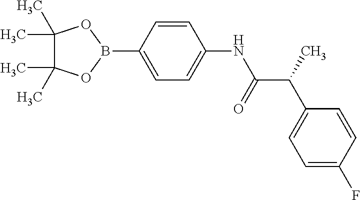 [SMILES: C[C@@H](C(=O)Nc1ccc(B2OC(C)(C)C(C)(C)O2)cc1)c1ccc(F)cc1]

To a stirred solution of 4-(4,4,5,5-tetramethyl-1,3,2-dioxaborolan-2-yl)aniline (1.0 g; CAS-RN: [8017-16-1]; >83% phosphate (as P₂O₅) from Sigma-Aldrich; Order No. 04101) in DMF (45 mL) and dichloromethane (90 mL) was added sodium bicarbonate (766 mg), (2R)-2-(4-fluorophenyl)propanoic acid (844 mg) and HATU (2.6 g). The mixture was stirred at room temperature for 4 h. Water was added, and the mixture was stirred for 30 minutes. A half-saturated solution of sodium bicarbonate was added and the mixture was extracted with ethyl acetate. The organic phase was washed with saturated sodium chloride solution, dried (sodium sulfate) and the solvent was removed in vacuum. Silica gel chromatography gave 1.53 g of the title compound.

¹H-NMR (400 MHz, DMSO-d₆), δ [ppm]=1.23 (12H), 1.37 (3H), 3.74-3.87 (1H), 7.06-7.16 (2H), 7.31-7.42 (2H), 7.51-7.61 (4H), 10.12 (1H).

### Intermediate Example Intl 1.05

### (4-{[(2R)-2-(4-fluorophenyl)propanoyl]amino}phenyl)boronic acid

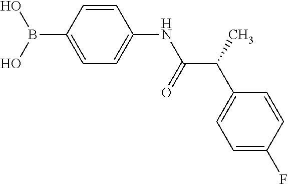 [SMILES: C[C@@H](C(=O)Nc1ccc(B(O)O)cc1)c1ccc(F)cc1]

To a stirred solution of (4-aminophenyl)boronic acid hydrochloride (2.00 g) in DMF (42 mL) was added sodium bicarbonate (2.9 g), (2R)-2-(4-fluorophenyl)propanoic acid (2.04 g) and HATU (6.58 g). The mixture was stirred at room temperature for 72 h. Water (140 mL) was added, and the mixture was stirred for 2 h. The white precipitate was collected by filtration and was washed with water and was dried in vacuum to give 2.86 g of the title compound.

¹H-NMR (300 MHz, DMSO-d₆), δ [ppm]=1.39 (3H), 3.84 (1H), 7.08-7.21 (2H), 7.35-7.44 (2H), 7.52 (2H), 7.69 (2H), 7.88 (2H), 10.07 (1H).

### Intermediate Example Intl 2.01

### 5-bromo-6-methoxy-2,3-dihydro-1-benzothiophene

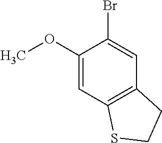

Int12.01 was prepared as described by David W. Robertson et al. in European Journal of Medicinal Chemistry, 1986, 21, p 223-229.

Int12.01 can also be prepared in a similar way as described below:

### Intermediate Example Intl 2.01.a

### 1-[(2,2-dimethoxyethyl)sulfanyl]-3-methoxybenzene

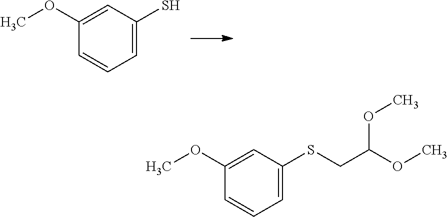

To a stirred solution of 3-methoxybenzenethiol (5.14 g) in acetonitrile (31 mL) was added potassium carbonate (6.08 g) and the mixture was stirred for 2 h at r.t. 2-Bromo-1,1-dimethoxyethane (7.67 g) was added and the mixture was stirred for at r.t. for 70 h. Water was added and the mixture was extracted with a mixture of ethyl acetate and hexane (1:1). The organic phase was dried (sodium sulfate) and the solvent was removed in vacuum. Silicagel chromatography gave 8.0 g of the title compound.

¹H-NMR (300 MHz, CHLOROFORM-d), δ [ppm]=3.15 (2H), 3.40 (6H), 3.82 (3H), 4.56 (1H), 6.76 (1H), 6.92-7.01 (2H), 7.19-7.26 (1H).

### Intermediate Example Intl 2.01.b

### 6-methoxy-1-benzothiophene

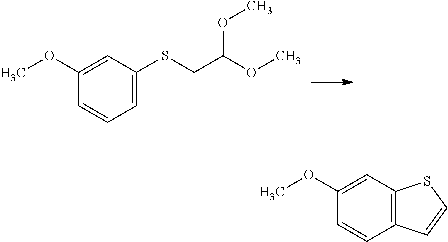

To a stirred solution of 1-[(2,2-dimethoxyethyl)sulfanyl]-3-methoxybenzene (1.0 g) in chlorobenzene (40 mL) was added polyphosphoric acid (1.0 g) and the mixture was heated to 80° C. for 1 h. The mixture was cooled to 0° C. with an ice-bath and an aqueous solution of sodium hydroxide was added with ice bath cooling until pH7 was reached. The mixture was extracted with dichloromethane, the organic phase was dried (sodium sulfate) and the solvent was removed in vacuum. Silicagel chromatography gave 407 mg of the title compound, containing approx. 20% of a second isomer. This mixture was used for the next step without further purification.

¹H-NMR (400 MHz, DMSO-d₆), δ [ppm]=3.81 (3H), 6.99 (1H), 7.31-7.35 (1H), 7.51 (1H), 7.56 (1H), 7.74 (1H). The product contains approx. 20% of a second isomer.

### Intermediate Example Intl 2.01.c

### 6-methoxy-1-benzothiophene 1,1-dioxide

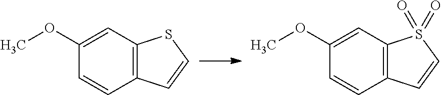

To a stirred solution of 6-methoxy-1-benzothiophene (700 mg) in chloroform (11 mL) at 0° C. was added 3-chlorobenzenecarboperoxoic acid (1.99 g) and the mixture was stirred for 2 h at r.t. An aqueous solution of disodium sulfurothioate was added, the mixture was stirred for 30 minutes and was consecutively extracted with ethyl acetate and with dichloromethane. Both organic phases were washed with a half saturated sodium bicarbonate solution and with saturated sodium chloride solution. The organic phases were combined, dried (sodium sulfate) and the solvent was removed in vacuum. Silicagel chromatography gave 612 mg of the title compound, containing approx. 20% of a second isomer. This mixture was used for the next step without further purification.

¹H-NMR (400 MHz, DMSO-d₆), δ [ppm]=3.86 (3H), 7.15-7.22 (2H), 7.45 (1H), 7.49 (1H), 7.54 (1H).

### Intermediate Example Intl 2.01.d

### 6-methoxy-2,3-dihydro-1-benzothiophene 1,1-dioxide

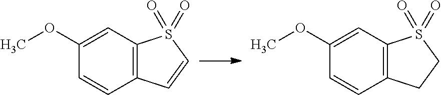

To a stirred solution of 6-methoxy-1-benzothiophene 1,1-dioxide (605 mg) in ethanol (10 mL) and dichloromethane (10 mL) was added palladium on carbon (10% w/w palladium) (147 mg) and the mixture was stirred at r.t. in a hydrogen atmosphere for 16 h. The mixture was filtered, and concentrated in vacuum. Silicagel chromatography gave a solid that was recrystallized from ethanol to give 248 mg of the pure title compound.

¹H-NMR (300 MHz, DMSO-d₆), δ [ppm]=3.20-3.29 (2H), 3.53-3.63 (2H), 3.82 (3H), 7.18-7.25 (2H), 7.42 (1H).

### Intermediate Example Intl 2.01.e

### 6-methoxy-2,3-dihydro-1-benzothiophene

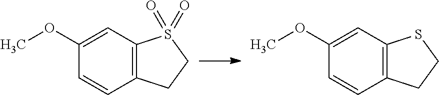

To a stirred solution of 6-methoxy-2,3-dihydro-1-benzothiophene 1,1-dioxide (224 mg) in diethyl ether (80 mL) was added lithium aluminumhydride (386 mg) and the mixture was heated to reflux for 4 h. Water was added, and aqueous hydrochloric acid was added until a clear solution had formed. The mixture was extracted with diethyl ether, the solution was dried (sodium sulfate) and the solvent was removed in vacuum. Silica gel chromatography gave 136 mg of the title compound.

¹H-NMR (300 MHz, DMSO-d₆), δ [ppm]=3.08-3.17 (2H), 3.28-3.37 (2H), 3.69 (3H), 6.55 (1H), 6.81 (1H), 7.11 (1H).

### Intermediate Example Intl 2.01

### 5-bromo-6-methoxy-2,3-dihydro-1-benzothiophene

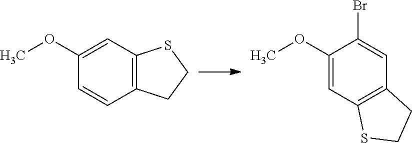

To a stirred solution of 6-methoxy-2,3-dihydro-1-benzothiophene (136 mg) in trichloromethane (9.5 mL) was added a freshly prepared solution of bromine in trichloromethane (0.44 mL; c=10% w/w) at 0° C. and the solution was stirred at 0° C. for 1 h. An aqueous solution of disodium sulfurothioate was added, and the mixture was extracted with dichloromethane. The organic phase was dried (sodium sulfate) and the solvent was removed in vacuum. Silica gel chromatography gave 170 mg of the title compound.

¹H-NMR (400 MHz, DMSO-d₆), δ [ppm]=3.13-3.19 (2H), 3.34-3.40 (2H), 3.78 (3H), 7.03 (1H), 7.33-7.45 (1H).

### Intermediate Example Intl 2.02

### 5-bromo-1,1-dioxido-2,3-dihydro-1-benzothiophen-6-yl methyl ether

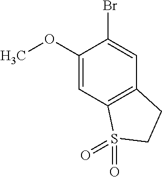

To a stirred solution of 5-bromo-6-methoxy-2,3-dihydro-1-benzothiophene (200 mg) in chloroform (15 mL) was added 3-chlorobenzenecarboperoxoic acid (380 mg) and the mixture was stirred for 1 h at r.t. An aqueous solution of disodium sulfurothioate was added, the mixture was stirred for 30 minutes and was extracted with dichloromethane. The organic phase was washed with a half saturated potassium carbonate solution and with saturated sodium chloride solution, dried (sodium sulfate) and the solvent was removed in vacuum. Silica gel chromatography gave 130 mg of the title compound.

¹H-NMR (400 MHz, DMSO-d₆), δ [ppm]=3.26 (2H), 3.59 (2H), 3.93 (3H), 7.40 (1H), 7.82 (1H).

### Intermediate Example Intl 2.03

### 6-chloro-N-(6-methoxy-1,1-dioxido-2,3-dihydro-1-benzothiophen-5-yl)[1,2,4]triazolo[1,5-a]pyridin-2-amine

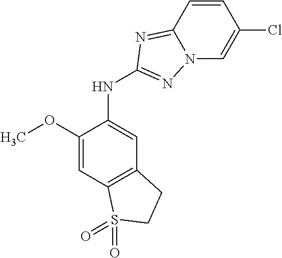 [SMILES: COc1cc2c(cc1Nc1nc3ccc(Cl)cn3n1)CCS2(=O)=O]

To a stirred suspension of 6-chloro[1,2,4]triazolo[1,5-a]pyridin-2-amine (68.7 mg) in toluene (2.8 mL) and NMP (0.17 mL) was added 5-bromo-1,1-dioxido-2,3-dihydro-1-benzothiophen-6-yl methyl ether (130 mg), chloro(2-dicyclohexylphosphino-2′,4′,6′-tri-isopropyl-1,1′-biphenyl)[2-(2-aminoethyl)phenyl] palladium(II) methyl-tert-butylether adduct (10.1 mg), X-Phos (5.95 mg) and powdered potassium phosphate monohydrate (303 mg). The flask was degassed twice and backfilled with argon. The mixture was heated to reflux for 1 h. The reaction mixture was filtered through an aminophase-silicagel column and the solvent was removed in vacuum. Silicagel chromatography gave 56 mg of the title compound.

¹H-NMR (300 MHz, DMSO-d₆), δ [ppm]=3.25-3.31 (2H), 3.50-3.61 (2H), 3.95 (3H), 7.29 (1H), 7.69 (2H), 8.32 (1H), 8.67 (1H), 9.22 (1H).

### Compounds of the Present Invention

### Example 01.01

### (2R)-2-(4-fluorophenyl)-N-(4-{2-[(6-methoxy-1,1-dioxido-2,3-dihydro-1-benzothiophen-5-yl)amino][1,2,4]triazolo[1,5-a]pyridin-6-yl}phenyl)propanamide

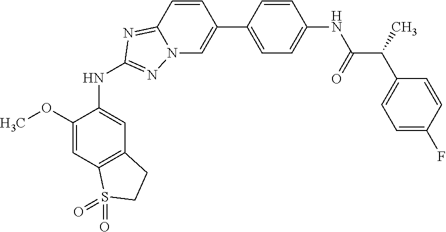 [SMILES: COc1cc2c(cc1Nc1nc3ccc(-c4ccc(NC(=O)[C@H](C)c5ccc(F)cc5)cc4)cn3n1)CCS2(=O)=O]

To a stirred suspension of Int12.03 (6-chloro-N-(6-methoxy-1,1-dioxido-2,3-dihydro-1-benzothiophen-5-yl)[1,2,4]triazolo[1,5-a]pyridin-2-amine) (53.0 mg) in toluene (1.1 mL) and NMP (0.1 mL) was added Int11.05 ((4-{[(2R)-2-(4-fluorophenyl)propanoyl]amino} phenyl)boronic acid) (62.6 mg), powdered potassium phosphate monohydrate (123 mg), dicyclohexyl(2′,6′-dimethoxybiphenyl-2-yl)phosphine (11.9 mg) and Pd₂dba₃ (6.65 mg) and the flask was degassed twice and backfilled with argon. The mixture was heated to reflux for 1 h. The reaction mixture was filtered through an aminophase-silicagel column and the solvent was removed in vacuum. Further aminophase-silicagel chromatography gave 30 mg of the title compound.

¹H-NMR (400 MHz, DMSO-d₆), δ [ppm]=1.42 (3H), 3.30-3.34 (2H), 3.51-3.59 (2H), 3.82-3.91 (1H), 3.97 (3H), 7.11-7.21 (2H), 7.29 (1H), 7.39-7.48 (2H), 7.66-7.79 (5H), 7.95 (1H), 8.38 (1H), 8.54 (1H), 9.14 (1H), 10.19 (1H).

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

### Biological Assay: Proliferation Assay

Cultivated tumor cells (MCF7, hormone dependent human mammary carcinoma cells, ATCC HTB22; NCI-H460, human non-small cell lung carcinoma cells, ATCC HTB-177; DU 145, hormone-independent human prostate carcinoma cells, ATCC HTB-81; HeLa-MaTu, human cervical carcinoma cells, EPO-GmbH, Berlin; HeLa-MaTu-ADR, multidrug-resistant human cervical carcinoma cells, EPO-GmbH, Berlin; HeLa human cervical tumor cells, ATCC CCL-2; B16F10 mouse melanoma cells, ATCC CRL-6475) were plated at a density of 5000 cells/well (MCF7, DU145, HeLa-MaTu-ADR), 3000 cells/well (NCI-H460, HeLa-MaTu, HeLa), or 1000 cells/well (B16F10) in a 96-well multititer plate in 200 μl of their respective growth medium supplemented 10% fetal calf serum. After 24 hours, the cells of one plate (zero-point plate) were stained with crystal violet (see below), while the medium of the other plates was replaced by fresh culture medium (200 μl), to which the test substances were added in various concentrations (0 μM, as well as in the range of 0.01-30 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were stained by adding 100 μl/measuring point of a 0.1% crystal violet solution (pH 3.0). After three washing cycles of the stained cells with water, the plates were dried at room temperature. The dye was dissolved by adding 100 μl/measuring point of a 10% acetic acid solution. The extinction of the stained cells was determined by photometry at a wavelength of 595 nm. The change of cell number, in percent, was calculated by normalization of the measured values to the extinction values of the zero-point plate (=0%) and the extinction of the untreated (0 μm) cells (=100%). The 1050 values were determined by means of a 4 parameter fit using the company's own software.

The compounds of the present invention are characterized by the following 1050 values, determined in a HeLa cell proliferation assay (as described above):

| | Inhibition of cell proliferation, |
|---|---|
| | cell Line: HeLa |
| Example Number | IC₅₀ |
| Example01.01 | <400 nM |

### Mps-1 Kinase Assay

The human kinase Mps-1 phosphorylates a biotinylated substrate peptide. Detection of the phosphorylated product is achieved by time-resolved fluorescence resonance energy transfer (TR-FRET) from Europium-labelled anti-phospho-Serine/Threonine antibody as donor to streptavidin labelled with cross-linked allophycocyanin (SA-XLent) as acceptor. Compounds are tested for their inhibition of the kinase activity.

N-terminally GST-tagged human full length recombinant Mps-1 kinase (purchased from Invitrogen, Karslruhe, Germany, cat. no PV4071) was used. As substrate for the kinase reaction a biotinylated peptide of the amino-acid sequence PWDPDDADITEILG (C-terminus in amide form, purchased from Biosynthan GmbH, Berlin) was used.

For the assay 50 nl of a 100-fold concentrated solution of the test compound in DMSO was pipetted into a black low volume 384 well microtiter plate (Greiner Bio-One, Frickenhausen, Germany), 2 μl of a solution of Mps-1 in assay buffer [0.1 mM sodium-ortho-vanadate, 10 mM MgCl₂, 2 mM DTT, 25 mM Hepes pH 7.7, 0.05% BSA, 0.001% Pluronic F-127] were added and the mixture was incubated for 15 min at 22° C. to allow pre-binding of the test compounds to Mps-1 before the start of the kinase reaction. Then the kinase reaction was started by the addition of 3 μl of a solution of 16.7 adenosine-tri-phosphate (ATP, 16.7 μM=>final conc. in the 5 μl assay volume is 10 μM) and peptide substrate (1.67 μM=>final conc. in the 5 μl assay volume is 1 μM) in assay buffer and the resulting mixture was incubated for a reaction time of 60 min at 22° C. The concentration of Mps-1 in the assay was adjusted to the activity of the enzyme lot and was chosen appropriate to have the assay in the linear range, typical enzyme concentrations were in the range of about 1 nM (final conc. in the 5 μl assay volume). The reaction was stopped by the addition of 3 μl of a solution of HTRF detection reagents (100 mM Hepes pH 7.4, 0.1% BSA, 40 mM EDTA, 140 nM Streptavidin-XLent [#61GSTXLB, Fa. Cis Biointernational, Marcoule, France], 1.5 nM anti-phospho(Ser/Thr)-Europium-antibody [#AD0180, PerkinElmer LAS, Rodgau-Jugesheinn, Germany].

The resulting mixture was incubated 1 h at 22° C. to allow the binding of the phosphorylated peptide to the anti-phospho(Ser/Thr)-Europium-antibody. Subsequently the amount of phosphorylated substrate was evaluated by measurement of the resonance energy transfer from the Europium-labelled anti-phospho(Ser/Thr) antibody to the Streptavidin-XLent. Therefore, the fluorescence emissions at 620 nm and 665 nm after excitation at 350 nm was measured in a Viewlux TR-FRET reader (PerkinElmer LAS, Rodgau-Jugesheinn, Germany). The “blank-corrected normalized ratio” (a Viewlux specific readout, similar to the traditional ratio of the emissions at 665 nm and at 622 nm, in which blank and Eu-donor crosstalk are subtracted from the 665 nm signal before the ratio is calculated) was taken as the measure for the amount of phosphorylated substrate. The data were normalised (enzyme reaction without inhibitor=0% inhibition, all other assay components but no enzyme=100% inhibition). Test compounds were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit using an inhouse software.

The compounds of the present invention are characterized by the following IC₅₀ values, determined in Mps-1 kinase assays (as described above):

| | Mps-1 Inhibition, | Mps-1 Inhibition, |
|---|---|---|
| | IC₅₀ | IC₅₀ |
| Example Number | (Assay with 10 μM ATP) | (Assay with 2 mM ATP) |
| Example01.01 | <1 nM | 1.6 nM |

Spindle Assembly Checkpoint Assay

The spindle assembly checkpoint assures the proper segregation of chromosomes during mitosis. Upon entry into mitosis, chromosomes begin to condensate which is accompanied by the phosphorylation of histone H3 on serine 10. Dephosphorylation of histone H3 on serine 10 begins in anaphase and ends at early telophase. Accordingly, phosphorylation of histone H3 on serine 10 can be utilized as a marker of cells in mitosis. Nocodazole is a microtubule destabilizing substance. Thus, nocodazole interferes with microtubule dynamics and mobilises the spindle assembly checkpoint. The cells arrest in mitosis at G2/M transition and exhibit phosphorylated histone H3 on serine 10. An inhibition of the spindle assembly checkpoint by Mps-1 inhibitors overrides the mitotic blockage in the presence of nocodazole, and the cells complete mitosis prematurely. This alteration is detected by the decrease of cells with phosphorylation of histone H3 on serine 10. This decline is used as a marker to determine the capability of compounds of the present invention to induce a mitotic breakthrough.

Cultivated cells of the human cervical tumor cell line HeLa (ATCC CCL-2) were plated at a density of 2500 cells/well in a 384-well microtiter plate in 20 μl Dulbeco's Medium (w/o phenol red, w/o sodium pyruvate, w 1000 mg/ml glucose, w pyridoxine) supplemented with 1% (v/v) glutamine, 1% (v/v) penicillin, 1% (v/v) streptomycin and 10% (v/v) fetal calf serum. After incubation overnight at 37° C., 10 μl/well nocodazole at a final concentration of 0.1 μg/ml were added to cells. After 24 h incubation, cells were arrested at G2/M phase of the cell cycle progression. Test compounds solubilised in dimethyl sulfoxide (DMSO) were added at various concentrations (0 μM, as well as in the range of 0.005 μM—10 μM; the final concentration of the solvent DMSO was 0.5% (v/v)). Cells were incubated for 4 h at 37° C. in the presence of test compounds. Thereafter, cells were fixed in 4% (v/v) paraformaldehyde in phosphate buffered saline (PBS) at 4° C. overnight then permeabilised in 0.1% (v/v) Triton X™ 100 in PBS at room temperature for 20 min and blocked in 0.5% (v/v) bovine serum albumin (BSA) in PBS at room temperature for 15 min. After washing with PBS, 20 μl/well antibody solution (anti-phospho-histone H3 clone 3H10, FITC; Upstate, Cat#16-222; 1:200 dilution) was added to cells, which were incubated for 2 h at room temperature. Afterwards, cells were washed with PBS and 20 μl/well HOECHST 33342 dye solution (5 μg/ml) was added to cells and cells were incubated 12 min at room temperature in the dark. Cells were washed twice with PBS then covered with PBS and stored at 4° C. until analysis. Images were acquired with a Perkin Elmer OPERA™ High-Content Analysis reader. Images were analyzed with image analysis software MetaXpress™ from Molecular devices utilizing the Cell Cycle application module. In this assay both labels HOECHST 33342 and phosphorylated Histone H3 on serine 10 were measured. HOECHST 33342 labels DNA and is used to count cell number. The staining of phosphorylated Histone H3 on serine 10 determines the number of mitotic cells. Inhibition of Mps-1 decreases the number of mitotic cells in the presence of nocodazole indicating an inappropriate mitotic progression. The raw assay data were further analysed by four parameter logistic regression analysis to determine the IC₅₀ value for each tested compound.

Thus the compounds of the present invention effectively inhibit Mps-1 kinase and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Mps-1, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

Determination of Metabolic Stability In Vitro

(including calculation of hepatic in vivo blood clearance (CL) and of maximal oral bioavailability (Fₘₐₓ))

The metabolic stability of test compounds in vitro was determined by incubating them at 1 μM with a suspension of liver microsomes in 100 mM phosphate buffer, pH7.4 (NaH₂PO₄xH₂O+Na₂HPO₄x2H₂O) at a protein concentration of 0.5 mg/mL and at 37° C. The reaction was activated by adding a co-factor mix containing 1.2 mg NADP, 3 IU glucose-6-phosphate dehydrogenase, 14.6 mg glucose-6-phosphate and 4.9 mg MgCl₂ in phosphate buffer, pH 7.4. Organic solvent in the incubations was limited to <0.2% dimethylsulfoxide (DMSO) and <1% methanol. During incubation, the microsomal suspensions were continuously shaken and aliquots were taken at 2, 8, 16, 30, 45 and 60 min, to which equal volumes of cold methanol were immediately added. Samples were frozen at −20° C. over night, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, specific liver weight and microsomal protein content the hepatic in vivo blood clearance (CL) and the maximal oral bioavailability (Fₘₐₓ) were calculated for the different species. The following parameter values were used: Liver blood flow—1.3 L/h/kg (human), 2.1 L/h/kg (dog), 4.2 L/h/kg (rat); specific liver weight—21 g/kg (human), 39 g/kg (dog), 32 g/kg (rat); microsomal protein content—40 mg/g.

With the described assay only phase-I metabolism of microsomes is reflected, e.g. typically oxidoreductive reactions by cytochrome P450 enzymes and flavin mono-oxygenases (FMO) and hydrolytic reactions by esterases (esters and amides).

The compounds of the present invention are characterized by the values of maximum oral bioavailability (Fₘₐₓ) in rat, dog and humans (determined by means of liver microsomes as described above) shown in the table below:

| | Rat liver | Human liver | Dog liver |
|---|---|---|---|
| Example | microsomes; | microsomes; | microsomes; |
| Number | Fmax [%] | Fmax [%] | Fmax [%] |
| Example01.01 | 96 | 83 | 65 |

1. A compound of formula (I): 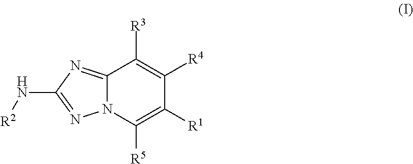
in which:
R¹ represents a phenyl- or a pyridyl- group;
which is substituted, one or more times, identically or differently, with a substituent selected from:
R⁶—(C₁-C₆-alkoxy)-, R⁶—O—, —C(═O)R⁶, —C(═O)O—R⁶, —N(H)C(═O)R⁶, —N(H)C(═O)NR⁶R⁷, —NR⁶R⁷, —C(═O)N(H)R⁶, —C(═O)NR⁶R⁷, R⁶—S—, R⁶—S(═O)₂—, —N(H)S(═O)₂R⁶, and —S(═O)₂N(H)R⁶; and
which is optionally substituted, one or more times, identically or differently, with a substituent selected from:
halo-, hydroxy-, nitro-, C₁-C₆-alkyl-, C₁-C₆-alkoxy-, hydroxy-C₁-C₆-alkyl-, —N(H)C(═O)R⁸, —N(H)C(═O)NR⁸R⁷, —C(═O)N(H)R⁸, and —N(H)S(═O)₂R⁸;
R² represents: 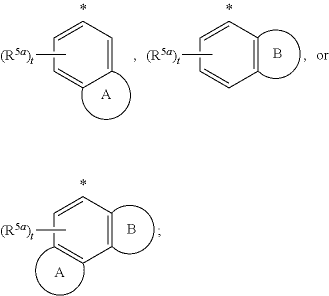
wherein * indicates the point of attachment of said group with the rest of the molecule;
A represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, or (C₃-C₆-cycloalkyl)-(CH₂)ₙ—O—;
B represents a 4- to 6-membered heterocyclic ring; which is optionally substituted, one or more times, identically or differently, with halo-, —CN, —OH, nitro-, C₁-C₆-alkyl-, halo-C₁-C₆-alkyl-, C₁-C₆-alkoxy-, halo-C₁-C₆-alkoxy-, hydroxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, or (C₃-C₆-cycloalkyl)-(CH₂)ₙ—O—;
R³ represents a hydrogen atom;
R⁴ represents a hydrogen atom;
R⁵ represents a hydrogen atom or a C₁-C₃-alkyl- group;
each
R^{5a} independently represents a group selected from:
halo-, nitro-, C₁-C₆-alkyl-, halo-C₁-C₆-alkyl-, C₁-C₆-alkoxy-, hydroxy-C₁-C₆-alkyl-, R⁸—(C₁-C₆-alkoxy)-, R⁸—O—, —NR⁸R⁷, R⁸—S—, R⁸—S(═O)—, R⁸—S(═O)₂—, and (C₃-C₆-cycloalkyl)-(CH₂)ₙ—O—;
R⁶ represents a group selected from:
C₃-C₆-cycloalkyl-, 3- to 10-membered heterocycloalkyl-,
aryl-, heteroaryl-, —(CH₂)_{q}—(C₃-C₆-cycloalkyl), —(CH₂)_{q}-(3- to 10-membered heterocycloalkyl), —(CH₂)_{q}-aryl and —(CH₂)_{q}-heteroaryl;
wherein said group being optionally substituted, one or more times, identically or differently, with a substituent selected from:
halo-, hydroxy-, cyano-, nitro-, C₁-C₆-alkyl-, halo C₁-C₆ alkyl, hydroxy-C₁-C₆-alkyl-, —C₆-alkoxy-C₁-C₆-alkyl-, —C₆-alkoxy-C₁-C₆-alkyl-, R⁸—(CH₂)ₙ(CHOH)(CH₂)ₘ—, R⁸—(C₁-C₆-alkoxy)-, R⁸—(CH₂)ₙ(CHOH)(CH₂)ₚ—O—, R⁸—(C₁-C₆-alkoxy-C₁-C₆-alkyl)-O—, aryl-, R⁸—O—, —C(═O)R⁸, —C(═O)O—R⁸, —OC(═O)—R⁸, —N(H)C(═O)R⁸, —N(R⁷)C(═O)R⁸, —N(H)C(═O)NR⁸R⁷, —N(R⁷)C(═O)NR⁸R⁷, —N(H)R⁸, —NR⁸R⁷, —C(═O)N(H)R⁸, —C(═O)NR⁸R⁷, R⁸—S(═O)—, R⁸—S(═O)₂—, —N(H)S(═O)R⁸, —N(R⁷)S(═O)R⁸, —S(═O)N(H)R⁸, —S(═O)NR⁸R⁷, —N(H)S(═O)₂R⁸, —N(R⁷)S(═O)₂R⁸, —S(═O)₂N(H)R⁸, —S(═O)₂NR⁸R⁷, —S(═O)(═NR⁸)R⁷, —S(═O)(═NR⁷)R⁸, and —N═S(═O)(R⁸)R⁷;
R⁷ represents a C₁-C₃-alkyl- or a C₃-C₆-cycloalkyl- group;
R⁸ represents a hydrogen atom or a C₁-C₆-alkyl- or C₃-C₆-cycloalkyl- group;
wherein said C₁-C₆-alkyl- or C₃-C₆-cycloalkyl- group is optionally substituted, one or more times, identically or differently, with a substituent selected from:
halo-, hydroxy-, —NHR⁷, —NR⁷R⁷, —N(C₁-C₃-alkyl)-C(═O)R⁷, —N(C₁-C₃-alkyl)-C(═O)OR⁷, R⁷—S(═O)₂—, C₁-C₃-alkoxy-, and halo —C₃-alkoxy-;
or
R⁷ and R⁸ together with the molecular fragment they are attached to represent a 4- to 6-membered heterocycloalkyl- group, which is optionally substituted, one or more times, identically or differently, with a halogen atom, a C₁-C₃-alkyl-, halo-C₁-C₃-alkyl- or C₁-C₃-alkoxy- group;
n, m, and p,
represent, independently from each other, an integer of 0, 1, 2 or 3;
q represents an integer of 0, 1, 2 or 3;
and
t represents an integer of 0, 1 or 2;
or a tautomer, an N-oxide, a hydrate, a solvate, or a salt thereof, or a mixture of same.

2. A compound according to claim 1, wherein:
t=1; and
R² represents: 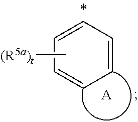
wherein * indicates the point of attachment of said group with the rest of the molecule.

3. A compound according to claim 1, wherein:
R² is selected from: 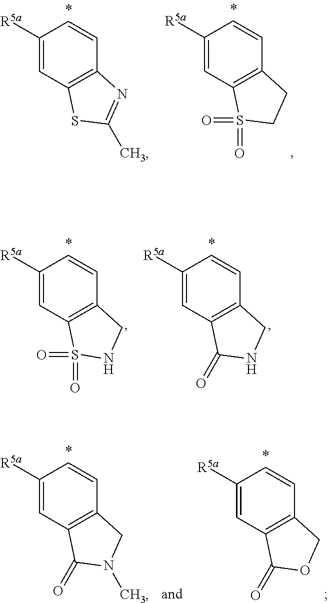
wherein * indicates the point of attachment of said groups with the rest of the molecule.

4. A compound according to claim 1, wherein:
R² represents: 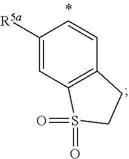
wherein * indicates the point of attachment of said groups with the rest of the molecule.

5. A compound according to claim 1, wherein:
R¹ represents 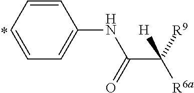
wherein * indicates the point of attachment of said group with the rest of the molecule;
wherein R^{6a} is a phenyl- group which is optionally substituted, one or more times, identically or differently, with a substituent selected from: halo-, methyl-, and methoxy-; and
wherein R⁹ represents a group selected from: C₁-C₃-alkyl-,
hydroxy-C₁-C₃-alkyl-, —N(R¹⁰)R¹⁰, and —C₁-C₂-alkyl-N(R¹⁰)R¹⁰; in which R¹⁰ represents a hydrogen atom or a methyl- group.

6. A compound according to claim 1, wherein:
R^{5a} represents a group selected from:
F-, methyl-, methoxy-, ethoxy-, n-propoxy-, iso-propoxy-,
cyclopropyl-O—, cyclopropyl-CH₂—O—, CH₃—O—CH₂CH₂—O—, CHF₂—O—, CF₃—O—, and CF₃CH₂—O—.

7. A compound according to claim 1, which is selected from the group consisting of: 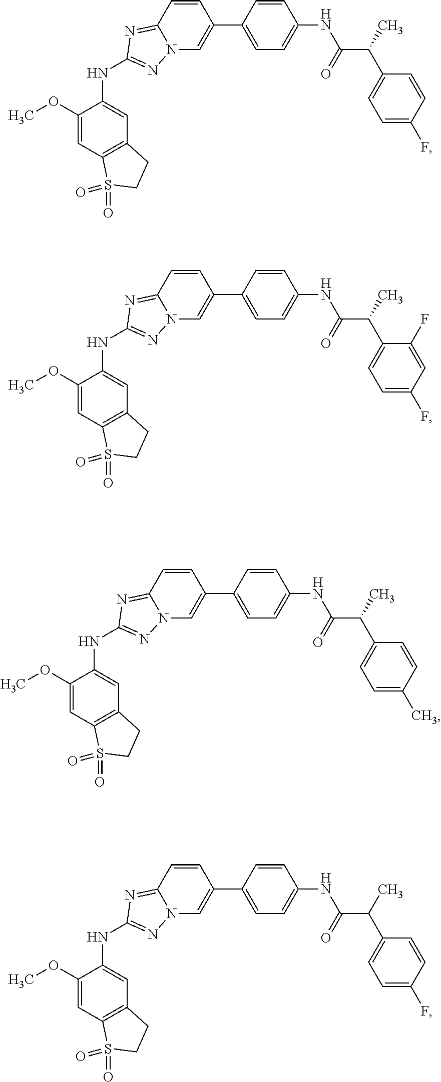 [SMILES: COc1cc2c(cc1Nc1nc3ccc(-c4ccc(NC(=O)[C@H](C)c5ccc(F)cc5)cc4)cn3n1)CCS2(=O)=O ; COc1cc2c(cc1Nc1nc3ccc(-c4ccc(NC(=O)[C@H](C)c5ccc(F)cc5F)cc4)cn3n1)CCS2(=O)=O ; COc1cc2c(cc1Nc1nc3ccc(-c4ccc(NC(=O)[C@H](C)c5ccc(C)cc5)cc4)cn3n1)CCS2(=O)=O ; COc1cc2c(cc1Nc1nc3ccc(-c4ccc(NC(=O)C(C)c5ccc(F)cc5)cc4)cn3n1)CCS2(=O)=O] 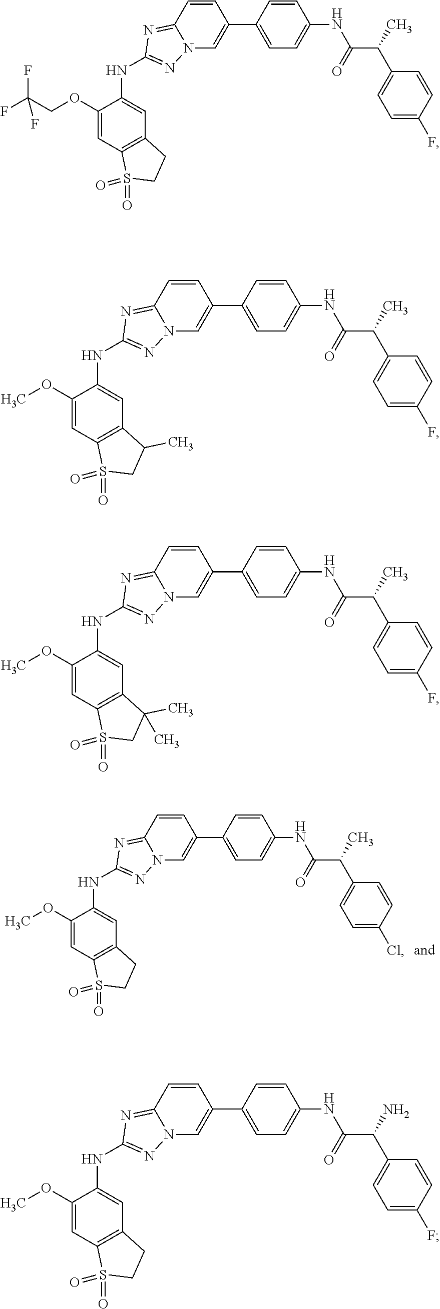 [SMILES: C[C@@H](C(=O)Nc1ccc(-c2ccc3nc(Nc4cc5c(cc4OCC(F)(F)F)S(=O)(=O)CC5)nn3c2)cc1)c1ccc(F)cc1 ; COc1cc2c(cc1Nc1nc3ccc(-c4ccc(NC(=O)[C@H](C)c5ccc(F)cc5)cc4)cn3n1)C(C)CS2(=O)=O ; COc1cc2c(cc1Nc1nc3ccc(-c4ccc(NC(=O)[C@H](C)c5ccc(F)cc5)cc4)cn3n1)C(C)(C)CS2(=O)=O ; COc1cc2c(cc1Nc1nc3ccc(-c4ccc(NC(=O)[C@H](C)c5ccc(Cl)cc5)cc4)cn3n1)CCS2(=O)=O ; COc1cc2c(cc1Nc1nc3ccc(-c4ccc(NC(=O)[C@H](N)c5ccc(F)cc5)cc4)cn3n1)CCS2(=O)=O]
or a tautomer, an N-oxide, a hydrate, a solvate, or a salt thereof, or a mixture of same.

8. (canceled)

9. (canceled)

10. (canceled)

11. A method for the treatment of a disease of uncontrolled cell growth, proliferation or survival, an inappropriate cellular immune response, or an inappropriate cellular inflammatory response, comprising administering to a patient in need thereof a therapeutically effective amount of a compound according to claim 1, or a tautomer, an N-oxide, a hydrate, a solvate, or a pharmaceutically acceptable salt thereof, or a mixture of same.

12. A method of preparing a compound of formula (I) according to claim 1, comprising reacting an intermediate compound of formula (5): 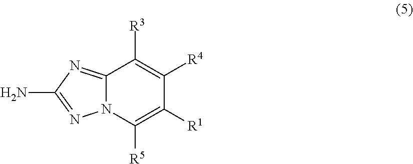
in which R¹, R³, R⁴, and R⁵ are as defined in claim 1,
with an aryl compound of formula (5a):
R²—Y (5a)
in which R² is as defined in claim 1, and Y represents a leaving group,
thus providing a compound of formula (I): 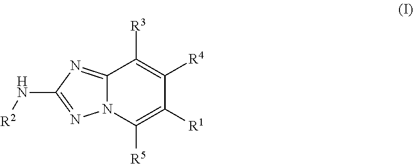
in which R¹, R², R³, R⁴, and R⁵ are as defined in claim 1.

13. A method of preparing a compound of general formula (I) according to claim 1, comprising reacting an intermediate compound of formula (7): 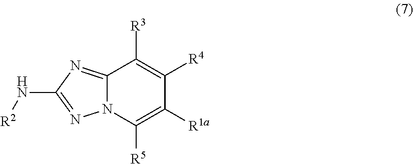
in which R², R³, R⁴, and R⁵ are as defined in claim 1, and R^{1a} is a phenyl group to which an —NH₂ substituent is bound in the para position,
with a compound of formula (7a):
R^{1b}—X (7a)
wherein R^{1b}—X represents 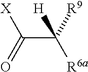
in which R⁹ and R^{6a} are as defined in claim 1, and X is a suitable functional group via which the R^{1b} of the compound of formula (7a) can be coupled, via a coupling reaction, onto the —NH₂ substituent bound to the phenyl group R^{1a} of compound (7),
thus providing a compound of formula (I): 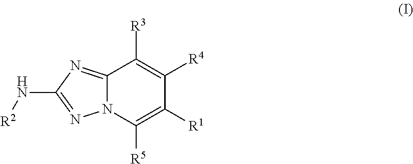
in which R¹, R², R³, R⁴, and R⁵ are as defined in claim 1.

14. A method of preparing a compound of formula (I) according to claim 1, comprising reacting an intermediate compound of formula (4): 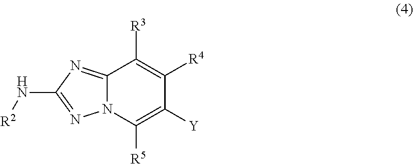
in which R², R³, R⁴, and R⁵ are as defined in claim 1, and Y represents a leaving group,
with a compound of formula (4a):
R¹—Z (4a)
in which R¹ is as defined in claim 1, and Z represents a boronic acid or a boronic ester,
thus providing a compound of formula (I): 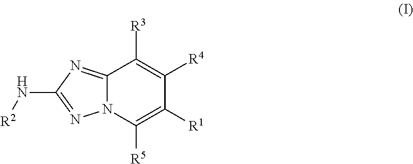
in which R¹, R², R³, R⁴, and R⁵ are as defined in claim 1.

15. (canceled)

16. A compound of formula (5a):
R²—Y (5a)
in which:
R² represents: 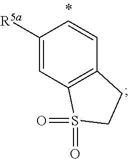
wherein * indicates the point of attachment of said group to Y;
R^{5a} represents a group selected from:
F-, methyl-, methoxy-, ethoxy-, n-propoxy-, iso-propoxy-,
cyclopropyl-O—, cyclopropyl-CH₂—O—, CH₃—O—CH₂CH₂—O—, CHF₂—O—, CF₃—O—, and CF₃CH₂—O—; and
Y represents a leaving group.

17. A compound of formula (5a):
R²—Y (5a)
in which:
R² represents: 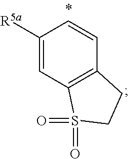
wherein * indicates the point of attachment of said group to Y;
R^{5a} represents a methoxy- group; and
Y represents a leaving group.

18. A compound of formula (4): 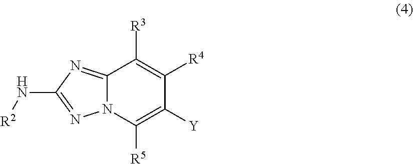
in which R², R³, R⁴, and R⁵ are as defined in claim 1, and Y represents a leaving group.

19. A compound of formula (4a):
R¹—Z (4a)
in which R¹ is as defined in claim 1, and Z represents a boronic acid or a boronic ester.

20. A compound of formula (5a):
R²—Y (5a)
in which R² is as defined in claim 1, and Y represents a leaving group.

21. A compound of formula (7a):
R^{1b}—X (7a)
wherein R^{1b}—X represents 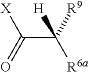
in which R⁹ and R^{6a} are as defined in claim 1, and X is a functional group via which the R^{1b} of the compound of formula (7a) can be coupled, via a coupling reaction, onto a —NH₂ substituent.

22. A compound of formula (7): 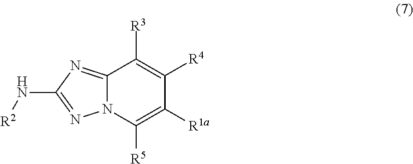
in which R², R³, R⁴, and R⁵ are as defined in claim 1, and R^{1a} is a phenyl group to which an —NH₂ substituent is bound in the para position.

23. The method according to claim 11, wherein the uncontrolled cell growth, proliferation or survival, inappropriate cellular immune response, or inappropriate cellular inflammatory response is mediated by Mps-1.

24. The method according to claim 23, wherein the disease of uncontrolled cell growth, proliferation or survival, inappropriate cellular immune response, or inappropriate cellular inflammatory response is a haemotological tumour, a solid tumour or metastases thereof.

25. The method according to claim 24, wherein the haemotological tumour, solid tumour or metastases thereof is selected from leukaemias and myelodysplastic syndrome, malignant lymphomas, head and neck tumours, brain tumours and brain metastases, tumours of the thorax, non-small cell and small cell lung tumours, gastrointestinal tumours, endocrine tumours, mammary and other gynaecological tumours, urological tumours, renal, bladder and prostate tumours, skin tumours, and sarcomas, or metastases thereof.


SEQUENCE LISTING (1 sequence)

SEQ ID NO: 1 - protein, 14 aa, Unknown
  biotinylated peptide of the amino-acid sequence PWDPDDADITEILG
PWDPDDADITEILG